SEQUENCE LISTING (26 sequences)

SEQ ID NO: 1 - protein, 9 aa, Artificial Sequence
  Description of Artificial Sequence Synthetic peptide
FTFNTYAMN

SEQ ID NO: 2 - protein, 19 aa, Artificial Sequence
  Description of Artificial Sequence Synthetic peptide
RIRSKYNNYATYYADSVKD

SEQ ID NO: 3 - protein, 14 aa, Artificial Sequence
  Description of Artificial Sequence Synthetic peptide
HGNFGNSYVSWFAY

SEQ ID NO: 4 - protein, 14 aa, Artificial Sequence
  Description of Artificial Sequence Synthetic peptide
RSSTGAVTTSNYAN

SEQ ID NO: 5 - protein, 7 aa, Artificial Sequence
  Description of Artificial Sequence Synthetic peptide
GTNKRAP

SEQ ID NO: 6 - protein, 9 aa, Artificial Sequence
  Description of Artificial Sequence Synthetic peptide
ALWYSNLWV

SEQ ID NO: 7 - protein, 9 aa, Artificial Sequence
  Description of Artificial Sequence Synthetic peptide
YTFTSYNMH

SEQ ID NO: 8 - protein, 17 aa, Artificial Sequence
  Description of Artificial Sequence Synthetic peptide
AIYPGNGDTSYNQKFKG

SEQ ID NO: 9 - protein, 12 aa, Artificial Sequence
  Description of Artificial Sequence Synthetic peptide
STYYGGDWYFNV

SEQ ID NO: 10 - protein, 10 aa, Artificial Sequence
  Description of Artificial Sequence Synthetic peptide
RASSSVSYIH

SEQ ID NO: 11 - protein, 7 aa, Artificial Sequence
  Description of Artificial Sequence Synthetic peptide
ATSNLAS

SEQ ID NO: 12 - protein, 9 aa, Artificial Sequence
  Description of Artificial Sequence Synthetic peptide
QQWTSNPPT

SEQ ID NO: 13 - protein, 15 aa, Artificial Sequence
  Description of Artificial Sequence Synthetic peptide
GGGGSGGGGSGGGGS

SEQ ID NO: 14 - protein, 5 aa, Artificial Sequence
  Description of Artificial Sequence Synthetic peptide
GGGGS

SEQ ID NO: 15 - protein, 357 aa, Artificial Sequence
  Description of Artificial Sequence Synthetic polypeptide
QAVVTQEPSLTVSPGGTVTLTCRSSTGAVTTSNYANWVQQKPGQAPRGLIGGTNKRAPGVPARFSGSLLGDKAALTLSGAQPEDEAEYYCALWYSNLWVFGTGTKVTVLGGGGSGGGGSGGGGSEVQLVESGGGLVQPGGSLKLSCAASGFTFNTYAMNWVRQASGKGLEWVARIRSKYNNYATYYADSVKDRFTISRDDSKNTLYLQMNSLKTEDTAVYYCVRHGNFGNSYVSWFAYWGQGTLVTVSSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSC

SEQ ID NO: 16 - protein, 354 aa, Artificial Sequence
  Description of Artificial Sequence Synthetic polypeptide
QIVLTQSPATLSLSPGERATLSCRASSSVSYIHWFQQKPGQAPRPWIYATSNLASGVPARFSGSGSGTDYTLTISSLEPEDFAVYYCQQWTSNPPTFGQGTKVEIKGGGGSGGGGSGGGGSQVQLVQSGAEVKKPGASVKVSCKASGYTFTSYNMHWVRQAPGQGLEWIGAIYPGNGDTSYNQKFKGKATLTADKSTSTAYMELSSLRSEDTAVYYCARSTYYGGDWYFNVWGQGTLVTVSSGGGGSRTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 17 - protein, 357 aa, Artificial Sequence
  Description of Artificial Sequence Synthetic polypeptide
EVQLVESGGGLVQPGGSLKLSCAASGFTFNTYAMNWVRQASGKGLEWVARIRSKYNNYATYYADSVKDRFTISRDDSKNTLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPGVPARFSGSLLGDKAALTLSGAQPEDEAEYYCALWYSNLWVFGTGTKVTVLGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSC

SEQ ID NO: 18 - protein, 354 aa, Artificial Sequence
  Description of Artificial Sequence Synthetic polypeptide
QVQLVQSGAEVKKPGASVKVSCKASGYTFTSYNMHWVRQAPGQGLEWIGAIYPGNGDTSYNQKFKGKATLTADKSTSTAYMELSSLRSEDTAVYYCARSTYYGGDWYFNVWGQGTLVTVSSGGGGSGGGGSGGGGSQIVLTQSPATLSLSPGERATLSCRASSSVSYIHWFQQKPGQAPRPWIYATSNLASGVPARFSGSGSGTDYTLTISSLEPEDFAVYYCQQWTSNPPTFGQGTKVEIKGGGGSRTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 19 - protein, 28 aa, Artificial Sequence
  Description of Artificial Sequence Synthetic peptide
GGGGSGGGGSGGGGSPKSCDKTHTCPPC

SEQ ID NO: 20 - protein, 495 aa, Artificial Sequence
  Description of Artificial Sequence Synthetic polypeptide
EVQLVESGGGLVQPGGSLKLSCAASGFTFNTYAMNWVRQASGKGLEWVARIRSKYNNYATYYADSVKDRFTISRDDSKNTLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPGVPARFSGSLLGDKAALTLSGAQPEDEAEYYCALWYSNLWVFGTGTKVTVLGGGGSGGGGSGGGGSPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 488 aa, Artificial Sequence
  Description of Artificial Sequence Synthetic polypeptide
QVQLVQSGAEVKKPGASVKVSCKASGYTFTSYNMHWVRQAPGQGLEWIGAIYPGNGDTSYNQKFKGKATLTADKSTSTAYMELSSLRSEDTAVYYCARSTYYGGDWYFNVWGQGTLVTVSSGGGGSGGGGSGGGGSQIVLTQSPATLSLSPGERATLSCRASSSVSYIHWFQQKPGQAPRPWIYATSNLASGVPARFSGSGSGTDYTLTISSLEPEDFAVYYCQQWTSNPPTFGQGTKVEIKGGGGSGGGGSGGGGSPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 125 aa, Artificial Sequence
  Description of Artificial Sequence Synthetic polypeptide
EVQLVESGGGLVQPGGSLKLSCAASGFTFNTYAMNWVRQASGKGLEWVARIRSKYNNYATYYADSVKDRFTISRDDSKNTLYLQMNSLKTEDTAVYYCVRHGNFGNSYVSWFAYWGQGTLVTVSS

SEQ ID NO: 23 - protein, 109 aa, Artificial Sequence
  Description of Artificial Sequence Synthetic polypeptide
QAVVTQEPSLTVSPGGTVTLTCRSSTGAVTTSNYANWVQQKPGQAPRGLIGGTNKRAPGVPARFSGSLLGDKAALTLSGAQPEDEAEYYCALWYSNLWVFGTGTKVTVL

SEQ ID NO: 24 - protein, 121 aa, Artificial Sequence
  Description of Artificial Sequence Synthetic polypeptide
QVQLVQSGAEVKKPGASVKVSCKASGYTFTSYNMHWVRQAPGQGLEWIGAIYPGNGDTSYNQKFKGKATLTADKSTSTAYMELSSLRSEDTAVYYCARSTYYGGDWYFNVWGQGTLVTVSS

SEQ ID NO: 25 - protein, 106 aa, Artificial Sequence
  Description of Artificial Sequence Synthetic polypeptide
QIVLTQSPATLSLSPGERATLSCRASSSVSYIHWFQQKPGQAPRPWIYATSNLASGVPARFSGSGSGTDYTLTISSLEPEDFAVYYCQQWTSNPPTFGQGTKVEIK

SEQ ID NO: 26 - protein, 13 aa, Homo sapiens
PKSCDKTHTCPPC

(19) United States
(12) Patent Application Publication
(10) Pub. No.: US 2020/0392230 A1
(43) Pub. Date: Dec. 17, 2020

(54) BISPECIFIC ANTI-CD3 X CD20 ANTIBODIES AND USES THEREOF

(71) Applicant: QLB Biotherapeutics, Boston, MA (US)

(72) Inventors: Chao TU, Belmont, MA (US); Rijian WANG, Saugus, MA (US)

(73) Assignee: QLB Biotherapeutics

(21) Appl. No.: 15/930,933

(22) Filed: May 13, 2020

Related U.S. Application Data
(60) Provisional application No. 62/847,383, filed on May 14, 2019.

(51) Int. Cl. C07K 16/28; A61P 35/00

(52) U.S. Cl. CPC C07K 16/2809; C07K 16/2887; A61P 35/00; A61K 2039/505; C07K 2317/74; C07K 2317/732; C07K 2317/31

(57) ABSTRACT

The disclosure relates to bispecific anti-CD3×CD20 antibodies comprising (i) a single-chain CD3 binding moiety, (ii) a single-chain CD20 binding moiety, and preferably (iii) an Fc region. Aspects of the disclosure further relate to compositions comprising these bispecific antibodies, vectors comprising one or more polynucleotides encoding these bispecific antibodies, as well as uses of these bispecific antibodies for treating a subject suffering from a B cell-related cancer, in particular, a subject who has developed resistance to anti-CD20, e.g., rituximab, therapy.

### CROSS-REFERENCE

This application claims the benefit of U.S. Provisional Application No. 62/847,383, filed May 14, 2019, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on May 13, 2020, is named 15007_0002-00304_SL.txt and is 30,162 bytes in size.

### FIELD

The disclosure relates to bispecific anti-CD3×CD20 antibodies, vectors comprising one or more polynucleotides encoding these bispecific antibodies, compositions comprising these bispecific antibodies, as well as uses of these bispecific antibodies. In some embodiments, the uses comprise treating a subject suffering from a B cell-related cancer, in particular, a subject who has developed resistance to anti-CD20, e.g., rituximab, therapy.

### BACKGROUND

Cancer is the second leading cause of death and is responsible for an estimated 9.6 million deaths in 2018. Globally, about 1 in 6 deaths is due to cancer.

Immunotherapy is one option for treating cancer. Several monoclonal antibodies (mAb) have received regulatory approval for treating cancers by blocking signaling pathways and/or inducing apoptosis in cancer cells. Despite these successes, however, the limitations of mAb cancer therapy remain. For example, many patients who initially respond to mAb treatment eventually relapse and/or develop treatment resistance.

To overcome the shortcomings of current mAb therapies and to improve treatment effectiveness, bispecific antibodies (BsAb) have been developed. BsAbs can simultaneously target two different antigens. Thus, BsAbs can be designed, for example, to block two different overlapping cellular pathways in cancer cells to potentially avoid or delay the development of treatment resistance. Alternatively, or in addition, BsAbs can be designed to target both the cancer cell and, for example, cells of the immune system, thereby directing specific immune cells to the tumor site to facilitate tumor killing. Many techniques for constructing BsAbs have emerged, including quadroma, knob-into-hole (KIH), nano-body, single-chain variable domain (scFv), and chemical cross-linking technologies.

Based on the types of biological targets and modes of action, bispecific immunotherapies can be divided into three main categories: (1) cytotoxic effector cell redirectors, including T-cell redirectors and natural killer (NK)-cell redirectors; (2) tumor-targeted immunomodulators; and (3) dual immunomodulators. Among these concepts, T-cell redirecting therapies are by far the most advanced. Indeed, two T-cell redirecting Abs, blinatumomab (Blincytoe) and catumaxomab (Removab®), have already reached the market, although the latter was withdrawn from the market in June 2017 due to insolvency of the manufacturer of the drug substance. In addition, more than twenty-five T-cell redirecting Abs targeting hematological and solid tumors are currently in phase I/II clinical trials.

Several T-cell redirected BsAbs have been developed in which one antigen-binding site targets the CD3 receptor to activate cytotoxic T lymphocytes and the other antigen-binding site targets tumor specific antigens including CD19, CD20, CD33, CD123, HER2, epithelial cell adhesion molecule (EpCAM), B cell maturation antigen (BCMA), carcino-embryonic antigen (CEA), and others. The convergence of cytotoxic T lymphocytes and tumor cells due to dual-binding by the BsAb activates cytotoxic T cells and promotes the destruction of tumor cells. Certain anti-CD3×CD20 BsAbs, including R07082859, XmAb13676, RG7828 (BTCT4465A) and FBTA05, are currently in phase I clinical trials.

The pan T cell surface marker CD3 plays a role in T cell activation. It serves as the signal transduction part of the T cell receptor (TCR)/CD3 complex. Under physiological conditions, when the TCR on T cells engages with the major histocompatibility complex (MHC)/antigen peptide on antigen presenting cells (APC), CD3 on T cells sends signals to initiate expression of an array of genes, resulting in T cell activation. Under experimental conditions, when CD3 molecules on T cells undergo cross-linking by mAbs, the cross-linked CD3 molecules will send similar signals to activate T cells. These properties make CD3 a promising target in BsAbs to engage and activate T cells to facilitate cancer cell destruction.

The pan B cell surface marker CD20 is a membrane glycoprotein that is over-expressed on most hematological malignancies in B cell linages. Thus, CD20 is a promising target for BsAbs designed to treat B cell leukemia and B cell lymphomas. Rituximab (Rituxan®), an anti-CD20 mAb, has emerged as a useful therapy for follicular non-Hodgkin Lymphoma (NHL). Rituximab is routinely incorporated into phases of conventional cancer treatment, including first-line therapy, maintenance, and salvage therapy, and has been used in hematopoietic cell transplantation (HCT) for follicular NHL. Rituximab has become an effective and widely used therapeutic mAb for treating such cancers. Nonetheless, as with many anti-tumor agents, the effectiveness of rituximab is ultimately limited, in part, by the development of treatment resistance.

Accordingly, there remains a need for novel anti-CD3×CD20 BsAbs that effectively treat a subject suffering from a B cell-related cancer, in particular, a subject who has developed resistance to traditional anti-CD20, e.g., rituximab, therapy.

### SUMMARY

The disclosure relates to bispecific anti-CD3×CD20 antibodies comprising (i) a single-chain CD3 binding moiety and (ii) a single-chain CD20 binding moiety. In some embodiments, the BsAbs disclosed herein further comprise (iii) an Fc region. Aspects of the disclosure further relate to compositions comprising these bispecific antibodies, vectors comprising one or more polynucleotides encoding these bispecific antibodies, as well as uses of these bispecific antibodies for treating a subject. In some embodiments, the subject suffers from a B cell-related cancer. In some embodiments, the subject has developed resistance to anti-CD20, e.g., rituximab, therapy.

Certain embodiments of the present disclosure are summarized in the following paragraphs. This list is only exemplary and not exhaustive of all of the embodiments provided by this disclosure.

Embodiment 1. A bispecific antibody comprising:

(i) a single-chain CD3 binding moiety comprising a heavy chain variable domain (VH) operably linked to a light chain variable domain (VL) via a first peptide linker, wherein

the VH comprises a heavy chain CDR1 comprising SEQ ID NO: 1, a heavy chain CDR2 comprising SEQ ID NO: 2, and a heavy chain CDR3 comprising SEQ ID NO: 3; and

the VL comprises a light chain CDR1 comprising SEQ ID NO: 4, a light chain CDR2 comprising SEQ ID NO: 5, and a light chain CDR3 comprising SEQ ID NO: 6, and

(ii) a single-chain CD20 binding moiety comprising a VH operably linked to a VL via a second peptide linker, wherein

the VH comprises a heavy chain CDR1 comprising SEQ ID NO: 7, a heavy chain CDR2 comprising SEQ ID NO: 8, and a heavy chain CDR3 comprising SEQ ID NO: 9;

and

the VL comprises a light chain CDR1 comprising SEQ ID NO: 10, a light chain CDR2 comprising SEQ ID NO: 11, and a light chain CDR3 comprising SEQ ID NO: 12.

Embodiment 2. The bispecific antibody of embodiment 1, wherein

the VH in the single-chain CD3 binding moiety comprises SEQ ID NO: 22, and the VL in the single-chain CD3 binding moiety comprises SEQ ID NO: 23; and/or

the VH in the single-chain CD20 binding moiety comprises SEQ ID NO: 24, and the VL in the single-chain CD20 binding moiety comprises SEQ ID NO: 25.

Embodiment 3. The bispecific antibody of embodiment 1 or 2, wherein the first and/or second peptide linker comprises SEQ ID NO: 13.

Embodiment 4. The bispecific antibody of any one of embodiments 1-3, further comprising a CH1 domain and a CK domain, wherein the CH1 domain and the CK domain forms a heterodimer.

Embodiment 5. The bispecific antibody of embodiment 4, wherein the VH in the single-chain CD3 binding moiety is operably linked to the CH1 domain via a third peptide linker, and wherein the VH in the single-chain CD20 binding moiety is operably linked to the CK domain via a fourth peptide linker.

Embodiment 6. The bispecific antibody of embodiment 5, wherein the third and/or fourth peptide linker comprises SEQ ID NO: 14.

Embodiment 7. The bispecific antibody of embodiment 6, comprising a first polypeptide sequence comprising SEQ ID NO: 15, and a second polypeptide comprising SEQ ID NO: 16.

Embodiment 8. The bispecific antibody of embodiment 4, wherein the VL in the single-chain CD3 binding moiety is operably linked to the CH1 domain via a third peptide linker, and wherein the VL in the single-chain CD20 binding moiety is operably linked to the CK domain via a fourth peptide linker.

Embodiment 9. The bispecific antibody of embodiment 8, wherein the third and/or fourth peptide linker comprises SEQ ID NO: 14.

Embodiment 10. The bispecific antibody of embodiment 9, comprising a first polypeptide sequence comprising SEQ ID NO: 17, and a second polypeptide comprising SEQ ID NO: 18.

Embodiment 11. The bispecific antibody of any one of embodiments 1-3, further comprising an Fc region comprising a first and second CH2 domain and a first and second CH3 domain.

Embodiment 12. The bispecific antibody of embodiment 11, wherein the VL in the single-chain CD3 binding moiety is operably linked to the first CH2 domain via a third peptide linker, and wherein the VL in the single-chain CD20 binding moiety is operably linked to the second CH2 domain via a fourth peptide linker.

Embodiment 13. The bispecific antibody of embodiment 12, wherein the third and/or fourth peptide linker comprises SEQ ID NO: 19.

Embodiment 14. The bispecific antibody of any one of embodiments 11-13, wherein the first and second CH2 domain comprises a L234A and/or L235A mutation.

Embodiment 15. The bispecific antibody of embodiment 14, comprising a first polypeptide sequence comprising SEQ ID NO: 20, and a second polypeptide comprising SEQ ID NO: 21.

Embodiment 16. An isolated polynucleotide encoding the single-chain CD3 binding moiety and/or the single-chain CD20 binding moiety of the bispecific antibody of any one of embodiments 1-15.

Embodiment 17. A vector comprising one or more polynucleotides encoding the bispecific antibody of any one of embodiments 1-15.

Embodiment 18. A composition comprising the bispecific antibody of any one of embodiments 1-15.

Embodiment 19. A pharmaceutical composition comprising the bispecific antibody of any one of embodiments 1-15 and a pharmaceutically acceptable carrier.

Embodiment 20. A host cell capable of expressing the bispecific antibody of any one of embodiments 1-15.

Embodiment 21. A method of producing a bispecific antibody, comprising culturing the host cell of embodiment 20 and recovering the bispecific antibody from the host cell.

Embodiment 22. A method of treating a subject suffering from a B cell-related cancer, comprising administrating to the subject a therapeutically effective amount of the bispecific antibody of any one of embodiments 1-15.

Embodiment 23. The method of embodiment 22, wherein the B cell-related cancer is selected from Hodgkin's lymphoma, non-Hodgkin's lymphoma, precursor B cell lymphoblastic leukemia/lymphoma, mature B cell neoplasms, B cell chronic lymphocytic leukemia, small lymphocytic lymphoma, B cell prolymphocytic leukemia, lymphoplasmacytic lymphoma, mantle cell lymphoma, follicular lymphoma, cutaneous follicle center lymphoma, marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma.

Embodiment 24. The method of any one of embodiments 22 and 23, wherein the subject has developed resistance to anti-CD20 therapy.

Embodiment 25. The method of any one of embodiments 22-24, wherein the subject has developed resistance to rituximab therapy.

Embodiment 26. The method of any one of embodiments 22-25, wherein the treatment results in significant or complete cancer remission.

### DESCRIPTION OF DRAWINGS

FIG. 1 depicts structural information for three exemplary anti-CD3×CD20 BsAbs of the disclosure: QLB1005-0001, QLB1005-0002, and QLB1005-0003.

FIG. 2 shows cell surface binding of QLB1005-0001, QLB1005-0002, QLB1005-0003, and isotype, SP34 (anti-CD3), and rituximab control antibodies to Jurkat and Raji cells, as assessed by a FACS analysis. EC50 and Emax values were shown in the tables. E is the effect at drug (i.e., antibody) concentration C, Emax is the maximal effect the drug can produce when all the receptors are occupied by the drug, and EC50 is the drug concentration that yields the half-maximal effect.

FIG. 3 shows functional activities of QLB1005-0001, QLB1005-0002, QLB1005-0003, and anti-CD3, anti-CD20, mixture of anti-CD3 and anti-CD20, and isotype control antibodies, as assessed by a luciferase reporter assay. Emax values were shown in the table.

FIG. 4 shows T cell proliferation induced by QLB1005-0001, QLB1005-0002, QLB1005-0003, and SP34 (anti-CD3), rituximab, and isotype control antibodies, as assessed by a FACS-based cell proliferation assay. EC50 values were shown in the tables.

FIG. 5 shows T cell proliferation induced by QLB1005-0002 and SP34 (anti-CD3), rituximab, and isotype control antibodies, as assessed by a FACS-based cell proliferation assay. QLB1005-0002 compositions produced from transient expression, CHOZN stable pool, and stable single clone 6A5 were labeled as QLB1005-0002, QLB1005-0002 (CHOZN), and QLB1005-0002 (6A5), respectively. EC50 values were shown in the tables.

FIG. 6 shows T cell killing of CD20⁺ tumor cells mediated by QLB1005-0001, QLB1005-0002, QLB1005-0003, and two isotype control antibodies, as assessed by a FACS analysis. The anti-CD3×CD20 BsAbs, but not control antibodies, showed dose-dependent killing activity in CD20⁺ target tumor cells (Raji and Daudi). EC50 values were shown in the tables.

FIG. 7 shows CD20 expression on rituximab resistant Raji and Daudi cells, as assessed by a FACS analysis.

FIG. 8 shows T cell killing activities on rituximab resistant Raji cells mediated by QLB1005-0001, QLB1005-0002, QLB1005-0003, and rituximab and isotype control antibodies. EC50 and Emax values were shown in the tables.

FIG. 9 shows T cell killing activities on rituximab resistant Raji cells mediated by QLB1005-0002 and rituximab and isotype control antibodies. QLB1005-0002 compositions produced from transient expression, CHOZN stable pool, and stable single clone 6A5 were labeled as QLB1005-0002, QLB1005-0002 (CHOZN), and QLB1005-0002 (6A5), respectively. EC50 and Emax values were shown in the tables.

FIGS. 10A and 10B show inhibition of Raji CDX tumor growth in hPBMC humanized NSG mice, mediated by QLB1005-0001, QLB1005-0002, QLB1005-0003, and vehicle control, respectively. Two groups of mice receiving hPBMCs from donor 1 (FIG. 10A) and donor 2 (FIG. 10B) were tested, and the tumor volume (TV) values were represented by mean±SEM.

FIG. 11 shows tumor volumes of individual animals post rechallenge with Raji cells mediated by QLB1005-0002 and vehicle control. 8 days post the last dose, the original Raji CDXs were removed by surgery. 4×10⁶ Raji cells were then inoculated s.c. in the left flanks of all 10 QLB1005-0002-treated mice and 5 vehicle control-treated mice. Rechallenged Raji CDX growth was measured 6 and 8-days post rechallenge.

FIGS. 12A and 12B show human T cell reconstitution in NSG mice.

Peripheral blood and spleen were collected at the end point and stained with antibodies specific for human CD45 and CD3. All mouse data were pooled together regardless of hPBMC donors. FIG. 12A shows reconstitution of human CD45 positive cells in mouse spleen and blood (mean±SEM). FIG. 12B shows percentage of human CD3 positive cells in human CD45 positive population in mouse spleen and blood (mean±SEM).

FIGS. 13A-13D show inhibition of rituximab resistant Raji CDX growth in hPBMC humanized NSG mice mediated by QLB1005-0002, vehicle control, and anti-CD3, rituximab, and mixture of anti-CD3 and rituximab control antibodies. FIG. 13A shows tumor growth curves of all treatment groups (mean±SEM). FIG. 13B shows an individual mouse tumor growth curve plotted for comparison between QLB1005-0002 and rituximab treated groups. FIG. 13C shows an individual mouse tumor volume on day 18 post treatment for all treatment groups. FIG. 13D shows human leucocyte reconstitution levels of all treatment groups on day 18 post treatment. Data of 3 mice in QLB1005-0002 and rituximab treated groups are shown; the other 3 mice in these 2 groups were kept for observation after day 18 post treatment.

FIG. 14 shows dose efficacy of QLB1005-0002 and vehicle control on Raji CDX growth in hPBMC humanized NSG mice. The plot shows the combined data from 2 hPBMC donors for each group (mean±SEM, n=10).

FIGS. 15A-15B show inhibition of B cell lymphoma line Daudi CDX growth in hPBMC humanized NSG mice, mediated by QLB1005-0002 and vehicle control. FIG. 15A shows tumor growth curves of vehicle control vs. QLB1005-0002 treated groups (mean±SEM). FIG. 15B shows a plot of an individual mouse tumor volume on day 17 post treatment.

FIG. 16 shows inhibition of Raji CDX growth in hPBMC humanized NSG mice mediated by QLB1005-0002 and vehicle control. The plot shows the combined data from two hPBMC donors for each group (mean±SEM, n=10).

### DETAILED DESCRIPTION

The disclosures and embodiments set forth herein are to be construed as exemplary only and not as limiting the scope of the invention. Although specific terms are employed herein, unless otherwise noted, they are used in a generic and descriptive sense only and not for purposes of limitation.

### Definitions

As used herein, the singular forms “a,” “an,” and “the” are intended to include the plural forms as well, unless the context clearly indicates otherwise.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has,” or “includes” one or more steps is not limited to possessing only those one or more steps and can also cover other unlisted steps. Similarly, any composition that “comprises,” “has,” or “includes” one or more features is not limited to possessing only those one or more features and can cover other unlisted features. The use of any and all examples, or exemplary language (e.g., “such as”) provided with respect to certain embodiments herein is intended merely to better illuminate the present disclosure and does not pose a limitation on the scope of the present disclosure otherwise claimed.

As used herein, the term “antibody” refers to any immunoglobulin (Ig) molecule, or any functional fragment, mutant, variant, or derivation thereof, that specifically binds to or interacts with at least one particular antigen (e.g., CD3 or CD20). Such mutant, variant, or derivative antibody formats are known in the art, and nonlimiting embodiments are discussed herein.

A full-length antibody includes immunoglobulin molecules comprising four polypeptide chains: two heavy (H) chains and two light (L) chains inter-connected by disulfide bonds. The heavy chains comprise a heavy chain variable region (HCVR or VH) and a heavy chain constant region. The heavy chain constant region comprises three domains, CH1, CH2, and CH3. The light chains comprise a light chain variable region (LCVR or VL) and a light chain constant region.

As used herein, the term “antigen-binding moiety” refers to an antibody fragment comprising one or more CDRs, or any other antibody fragment that binds to an antigen but does not comprise a full-length antibody structure (i.e., two full-length heavy chains and two full-length light chains). An antigen-binding moiety may be derived, e.g., from a full-length antibody molecule using any suitable standard techniques, such as proteolytic digestion or recombinant genetic engineering techniques known in the art. Examples of an antigen-binding moiety include, without limitation, a variable domain, a variable region, a Fab, and an Fv fragment. An antigen binding moiety is capable of binding to the same antigen to which the full-length antibody binds.

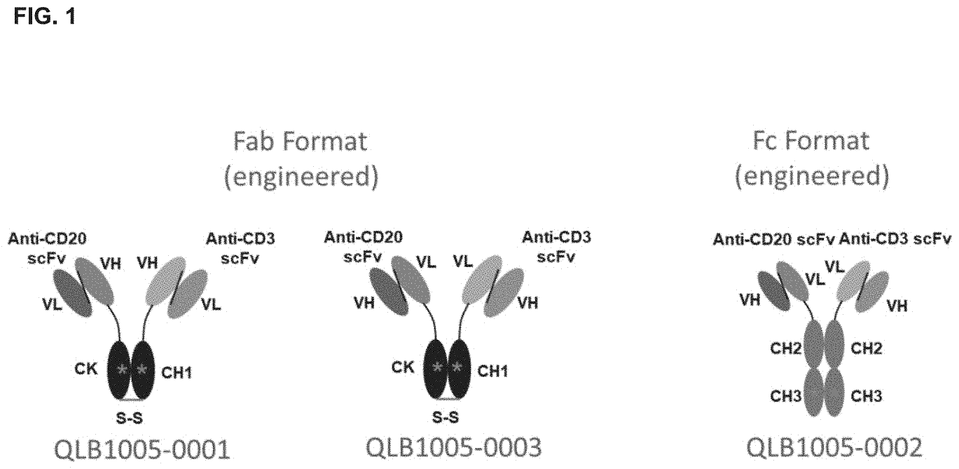

In some embodiments, an antibody may comprise an NCl/NIH CH1-CK stabilized Fab domain, as shown in FIG. 1 (QLB1005-0001 and QLB1005-0003). The NCl/NIH CH1-CK stabilized Fab format is known in the art (see, e.g., US 2018/0118815 A1; mAbs, 2016, 8(4):761-774). In some embodiments, an antibody may contain one or more domains from the constant region of a full-length antibody, for example, as shown in FIG. 1 (QLB1005-0002).

In some embodiments, the antibody according to the disclosure may be monospecific or multispecific (e.g., bispecific). In some embodiments, a multispecific antibody comprises at least two different variable domains, wherein each variable domain is capable of specifically binding to a separate antigen or to a different epitope on the same antigen. For example, in some embodiments a bispecific antibody (BsAb) may bind to two different antigens, for example, CD3 and CD20. In some embodiments, the bispecific antibodies are those shown in FIG. 1 that bind to both CD3 and CD20.

The VH and VL regions of an antibody can be further subdivided into regions of hypervariability, termed complementarity determining regions (CDRs), interspersed with regions that are more conserved, termed framework regions (FR). CDR boundaries for antibodies disclosed herein may be defined or identified by various known conventions, such as e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody. In general, the VH and VL are composed of three CDRs and four FRs, arranged from N-terminus to C-terminus in the following order: FR1, CDR1, FR2, CDR2, FR3, CDR3, and FR4.

As used herein, the terms “Fc” or “Fc region” refer to that portion of the antibody having the CH2 and CH3 of a first heavy chain bound to the CH2 and CH3 of a second heavy chain via disulphide bonding. The Fc region of an antibody is responsible for various effector functions such as ADCC and CDC, but does not function in antigen binding. In some embodiments, to reduce the potential effector function of the Fc region, L234A and L235A (LALA) mutations may be introduced to the CH2 domains of the Fc region (e.g., as shown in Example 1).

As used herein, the term “Fv” refers to the smallest fragment of the antibody to bear the complete antigen binding site. An Fv fragment contains the variable domain of a single light chain bound to the variable domain of a single heavy chain. A number of Fv designs are known in the art, including single chain Fv (“scFv”) which can be formed using a peptide linker to connect the VH and VL domains together as a single-chain polypeptide (e.g., as depicted in FIG. 1).

As used herein, the term “operably linked” refers to a juxtaposition, with a spacer or linker (e.g., a peptide linker according to this disclosure), of two or more biological sequences of interest in such a way that they are in a relationship permitting them to function in an intended manner. For example, an antibody VH region may be operably linked to a VL region so as to provide for a single-chain polypeptide sequence having antigen-binding activity.

As used herein, the terms “peptide linker” or “linker” refer to an amino acid sequence having 1, 2, 3, 4, 5, 10, 15, 20, 30, 40, or 50 amino acid residues joined by peptide bonds and used to link two or more polypeptides. Some representative peptide linker sequences are disclosed in Example 1.

As used herein, the terms “specifically bind” or “bind” to a particular antigen refer to binding that is measurably different from a non-specific interaction. Specific binding can be determined, for example, by comparing binding of a particular antibody to binding of an antibody that does not bind to a particular antigen. Specific binding for a particular antigen can be shown, for example, when an antibody has a Kd for an antigen of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁹ M, at least about 10⁻¹¹ M, at least about 10⁻¹² M, or greater, where Kd refers to a dissociation rate of the antibody/antigen interaction. In some embodiments, an antibody that specifically binds an antigen will have a Kd that is 20, 50, 100, 500, 1000, 5,000, 10,000 or more times greater than the Kd of an antibody that does not bind to the same antigen (e.g., an IgG molecule). In some embodiments, the binding between an antibody and a particular antigen can be shown by an EC50 value, determined using suitable methods known in the art, including, for example, flow cytometry assay. See, e.g., Example 1.

As used herein, the term “T cell” refers to a type of lymphocyte that plays a role in cell-mediated immunity, and includes helper T cells (e.g., CD4⁺ T cells, T helper 1 type T cells, T helper 2 type T cells, T helper 3 type T cells, T helper 17 type T cells), cytotoxic T cells (e.g., CD8⁺ T cells), memory T cells (e.g., central memory T cells), effector memory T cells, and resident memory T cells that are either CD8⁺ or CD4⁺, natural killer T (NKT) cells, and inhibitory T cells.

As used herein, the term “polynucleotide” refers to a polymer of deoxyribonucleic acids (DNA) or ribonucleic acids (RNA) in either single or double stranded form. Unless specifically limited, the term “polynucleotide” encompasses polynucleotides containing known analogues of natural nucleotides that have similar binding properties as the reference nucleic acid and are metabolized in a manner similar to naturally occurring nucleotides.

As used herein, the terms “vector” or “expression vector” refer to a vehicle into which a polynucleotide encoding a protein may be inserted so as to bring about the expression of that protein. In some embodiments, a “vector” includes appropriate regulatory sequences known in the art to control protein expression.

As used herein, the term “host cell” refers to a cell into which an exogenous polynucleotide and/or a vector has been introduced, such that the cell expresses a protein of interest. In some embodiments, the host cell expresses the anti-CD3×CD20 bispecific antibodies disclosed herein.

As used herein, the terms “treating” or “treatment” refer to preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

With regard to cancers or tumors, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. In some embodiments, the cancer is a B cell-related cancer selected from Hodgkin's lymphoma, non-Hodgkin's lymphoma, precursor B cell lymphoblastic leukemia/lymphoma, mature B cell neoplasms, B cell chronic lymphocytic leukemia, small lymphocytic lymphoma, B cell prolymphocytic leukemia, lymphoplasmacytic lymphoma, mantle cell lymphoma, follicular lymphoma, cutaneous follicle center lymphoma, marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma.

As used herein, the terms “administer” or “administering” refer to the placement of an antibody composition into a subject by a method or route that results in at least partial localization of the antibody composition at a desired site or tissue location. For example, an antibody composition may be administered to a subject by intramuscular injection, subcutaneous delivery, intranasal delivery, oral delivery, intradermal delivery, or transdermal delivery.

As used herein, the term “effective amount” refers to the amount of therapeutic agent, pharmaceutical composition, or pharmaceutical formulation, sufficient to reduce at least one or more symptom(s) of a disease or disorder, or to provide a desired effect.

As used herein, the term “subject” refers to an animal, for example a human, to whom treatment with methods and compositions described herein is provided.

Anti-CD3×CD20 Bispecific Antibodies

Bispecific antibodies (BsAbs) that target both CD3 and a tumor antigen can be designed and used to redirect T cells to attack and lyse the targeted tumor cells. In some embodiments, BsAbs according to this disclosure target both CD3 and CD20. In some embodiments, BsAbs according to this disclosure comprise a first antigen-binding moiety that binds to CD3 and a second antigen-binding moiety that binds to CD20. In some embodiments, BsAbs according to this disclosure comprise a first antigen-binding moiety that binds to CD20 and a second antigen-binding moiety that binds to CD3.

a) CD3 Binding Moieties

In some embodiments, the CD3 binding moiety is derived from humanized SP34 (ATCC) and contains the CDR sequences shown in Table 1.

TABLE 1
CDR sequences of an exemplary CD3 binding moiety

| | CDR1 | CDR2 | CDR3 |
|---|---|---|---|
| VH | SEQ ID NO: 1 | SEQ ID NO: 2 | SEQ ID NO: 3 |
| | FTFNTYAMN | RIRSKYNNYATYYADSVKD | HGNFGNSYVSWFAY |
| VL | SEQ ID NO: 4 | SEQ ID NO: 5 | SEQ ID NO: 6 |
| | RSSTGAVTTSNYA | GTNKRAP | ALVVYSNLWV |
| | N | | |

In some embodiments, the CD3 binding moiety provided herein comprises any suitable framework region (FR) sequences, so long as the CD3 binding moiety can specifically bind to CD3 expressed on a cell surface. Binding of the CD3 binding moiety to CD3 can be measured by methods known in the art, for example, flow cytometry assay. See Example 1.

In some embodiments, the CD3 binding moiety contains the following heavy and light chain variable region sequences.

VH Sequence of an Exemplary CD3 Binding Moiety:

(SEQ ID NO: 22)
EVQLVESGGGLVQPGGSLKLSCAASGFTFNTYAMNWVRQASGKGLEWVAR
IRSKYNNYATYYADSVKDRFTISRDDSKNTLYLQMNSLKTEDTAVYYCVR
HGNFGNSYVSWFAYWGQGTLVTVSS.

VL Sequence of an Exemplary CD3 Binding Moiety:

(SEQ ID NO: 23)
QAVVTQEPSLTVSPGGTVTLTCRSSTGAVTTSNYANWVQQKPGQAPRGLI
GGTNKRAPGVPARFSGSLLGDKAALTLSGAQPEDEAEYYCALWYSNLWVF
GTGTKVTVL.

In some embodiments, the CD3 binding moiety according to this disclosure has an scFv structure and comprises a VH operably linked to a VL via a peptide linker. In some embodiments, the peptide linker comprises

(SEQ ID NO: 13)
GGGGSGGGGSGGGGS.

In some embodiments, the CD3 binding moiety according to this disclosure has the structure of VL-linker-VH (from N- to C-terminus). See, e.g., QLB1005-0001 shown in FIG. 1. In some embodiments, the CD3 binding moiety according to this disclosure has the structure of VH-linker-VL (from N- to C-terminus). See, e.g., QLB1005-0002 and QLB1005-0003 shown in FIG. 1.

b) CD20 Binding Moieties

In certain embodiments, the CD20 binding moiety is derived from humanized rituximab (Roche) and contains the CDR sequences shown in Table 2.

TABLE 2
CDR sequences of an exemplary CD20 binding moiety

| | CDR1 | CDR2 | CDR3 |
|---|---|---|---|
| VH | SEQ ID NO: 7 | SEQ ID NO: 8 | SEQ ID NO: 9 |
| | YTFTSYNMH | AIYPGNGDTSYNQKFKG | STYYGGDWYFNV |
| VL | SEQ ID NO: 10 | SEQ ID NO: 11 | SEQ ID NO: 12 |
| | RASSSVSYIH | ATSNLAS | QQWTSNPPT |

In some embodiments, the CD20 binding moiety provided herein comprises any suitable framework region (FR) sequences, so long as the CD20 binding moiety can specifically bind to CD20 expressed on a cell surface. Binding of the CD20 binding moiety to CD20 can be measured by methods known in the art, for example, flow cytometry assay. See Example 1.

In some embodiments, the CD20 binding moiety contains the following heavy and light chain variable region sequences.

VH Sequence of an Exemplary CD20 Binding Moiety:

(SEQ ID NO: 24)
QVQLVQSGAEVKKPGASVKVSCKASGYTFTSYNMHWVRQAPGQGLEWIGA
IYPGNGDTSYNQKFKGKATLTADKSTSTAYMELSSLRSEDTAVYYCARST
YYGGDWYFNVWGQGTLVTVSS.

VL Sequence of an Exemplary CD20 Binding Moiety:

(SEQ ID NO: 25)
QIVLTQSPATLSLSPGERATLSCRASSSVSYIHWFQQKPGQAPRPWIYAT
SNLASGVPARFSGSGSGTDYTLTISSLEPEDFAVYYCQQWTSNPPTFGQG
TKVEIK.

In some embodiments, the CD20 binding moiety provided herein has an scFv structure and comprises a VH operably linked to a VL via a peptide linker. In some embodiments, the peptide linker comprises SEQ ID NO: 13.

In some embodiments, the CD20 binding moiety according to this disclosure has the structure of VL-linker-VH (from N- to C-terminus). See, e.g., QLB1005-0001 shown in FIG. 1. In some embodiments, the CD20 binding moiety according to this disclosure has the structure of VH-linker-VL (from N- to C-terminus). See, e.g., QLB1005-0002 and QLB1005-0003 shown in FIG. 1.

c) Anti-CD3×CD20 BsAbs

In some embodiments, the anti-CD3×CD20 BsAb according to this disclosure comprises an Fab format that comprises anti-CD3 scFV, CH1, anti-CD20 scFV, and CK domains.

In some embodiments, the anti-CD3×CD20 BsAb according to this disclosure comprises (i) a single chain CD3 binding moiety, (ii) a single chain CD20 binding moiety, and (iii) a CH1 domain and a CK domain, wherein the CH1 domain and the CK domain form a heterodimer via a disulphide bond. See FIG. 1, QLB1005-0001 and QLB1005-0003.

In some embodiments, the anti-CD3×CD20 BsAb comprises (i) a single-chain CD3 binding moiety comprising a VH operably linked to a VL via a first peptide linker, (ii) a single-chain anti-CD20 binding moiety comprising a VH operably linked to a VL via a second peptide linker, and (iii) a CH1 domain and a CK domain, wherein the VH in the single-chain anti-CD3 binding moiety is operably linked to the CH1 domain via a third peptide linker, the VH in the single-chain anti-CD20 binding moiety is operably linked to the CK domain via a fourth peptide linker, and the CH1 domain and the CK domain forms a heterodimer via a disulphide bond. See QLB1005-0001 shown in FIG. 1. In some embodiments, the first and/or second peptide linker comprises GGGGSGGGGSGGGGS (SEQ ID NO: 13). In some embodiments, the third and/or fourth peptide linker comprises GGGGS (SEQ ID NO: 14).

In some embodiments, the anti-CD3×CD20 BsAb of this disclosure comprises a first polypeptide sequence comprising SEQ ID NO: 15, and a second polypeptide sequence comprising SEQ ID NO: 16.

In some embodiments, the anti-CD3×CD20 BsAb comprises (i) a single-chain CD3 binding moiety comprising a VH operably linked to a VL via a first peptide linker, (ii) a single-chain anti-CD20 binding moiety comprising a VH operably linked to a VL via a second peptide linker, and (iii) a CH1 domain and a CK domain, wherein the VL in the single-chain anti-CD3 binding moiety is operably linked to the CH1 domain via a third peptide linker, the VL in the single-chain anti-CD20 binding moiety is operably linked to the CK domain via a fourth peptide linker, and the CH1 domain and the CK domain forms a heterodimer via a disulphide bond. See QLB1005-0003 shown in FIG. 1. In some embodiments, the first and/or second peptide linker comprises SEQ ID NO: 13. In some embodiments, the third and/or fourth peptide linker comprises SEQ ID NO: 14.

In some embodiments, the anti-CD3×CD20 BsAb of this disclosure comprises a first polypeptide sequence comprising SEQ ID NO: 17, and a second polypeptide sequence comprising SEQ ID NO: 18.

In some embodiments, the anti-CD3×CD20 BsAb of this disclosure comprises an IgG-like Fc format having a Y-shaped structure with two arms for antigen-binding and one stem for association and stabilization. In some embodiments, the anti-CD3×CD20 BsAb of the disclosure comprises an IgG-like Fc format comprising anti-CD3 scFV, anti-CD20 scFV, and an Fc region comprising a first and second CH2 domain and a first and second CH3 domain. In some embodiments, to reduce the potential effector function of the Fc region, L234A and L235A (LALA) mutations are introduced to the CH2 domains.

In some embodiments, the anti-CD3×CD20 BsAb of the disclosure comprises (i) a single-chain CD3 binding moiety comprising a VH operably linked to a VL via a first peptide linker, (ii) a single-chain anti-CD20 binding moiety comprising a VH operably linked to a VL via a second peptide linker, and (iii) an Fc region comprising a first and second CH2 domain and a first and second CH3 domain. See QLB1005-0002 shown in FIG. 1. In some embodiments, the VL in the single-chain CD3 binding moiety is operably linked to the first CH2 domain via a third peptide linker, and the VL in the single-chain CD20 binding moiety is operably linked to the second CH2 domain via a fourth peptide linker. In some embodiments, the first and/or second peptide linker comprises SEQ ID NO: 13. In some embodiments, the third and/or fourth peptide linker comprises GGGGSGGGGSGGGGSPKSCDKTHTCPPC (SEQ ID NO: 19). In some embodiments, the first and second CH2 domain comprises a L234A and/or L235A mutation.

In some embodiments, the anti-CD3×CD20 BsAb of the disclosure comprises a first polypeptide sequence comprising SEQ ID NO: 20, and a second polypeptide sequence comprising SEQ ID NO: 21.

Methods of Preparation

The present disclosure provides polynucleotide sequences that encode a single-chain CD3 binding moiety and/or a single-chain CD 20 binding moiety of the anti-CD3×CD20 BsAbs provided herein.

The polynucleotide sequences can be constructed using recombinant techniques known in the art. The polynucleotide sequences can be further operably linked to one or more regulatory sequences (promoters, origin of replication, selectable markers, ribosomal binding sites, inducers, etc.), preferably in an expression vector, such that the expression or production of the anti-CD3×CD20 BsAbs is feasible and controllable. In some embodiments, the expression vectors are extra-chromosomal. In some embodiments, the expression vectors are integrating vectors.

In some embodiments, the polynucleotide sequences and/or expression vectors according to this disclosure are introduced into a host cell for cloning or gene expression. Suitable host cells for cloning or expressing DNA are known in the art. In some embodiments, the host cell is a prokaryote, yeast, or higher eukaryote cell. Suitable prokaryotes for this purpose include eubacteria, such as Gram-negative or Gram-positive organisms, for example, Escherichia, e.g., E. coli. In addition to prokaryotes, eukaryotic microbes such as filamentous fungi or yeast are suitable cloning or expression hosts for the vectors encoding the anti-CD3×CD20 BsAbs disclosed herein. Vertebrate cells can also be used to express the vectors encoding the anti-CD3×CD20 BsAbs disclosed herein. In some embodiments, the vertebrate host cells lines are monkey kidney CV1 lines transformed by SV40 (e.g, COS-7, ATCC CRL 1651), human embryonic kidney line (e.g., 293 or 293 cells), baby hamster kidney cells (e.g., BHK, ATCC CCL 10), Chinese hamster ovary cells, monkey kidney cells (e.g., CV1 ATCC CCL 70), African green monkey kidney cells (e.g., VERO-76, ATCC CRL-1587), and canine kidney cells (e.g., MDCK, ATCC CCL 34).

Host cells transformed with the above-described polynucleotide sequences or vectors can be cultured in conventional nutrient media known in the art. For example, commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, and/or glucose or an equivalent energy source. Suitable culture conditions, such as temperature, pH, and the like, are known in the art.

In some embodiments, the present disclosure provides a method of producing the anti-CD3×CD20 BsAb disclosed herein, comprising culturing the host cell provided herein under suitable conditions for expressing the anti-CD3×CD20 BsAb. In certain embodiments, the method further comprises recovering the anti-CD3×CD20 BsAb from the host cell.

Once produced, the anti-CD3×CD20 BsAbs disclosed herin can be purified using techniques known in the art, for example, ion exchange chromatography, gel electrophoresis, dialysis, ammonium sulfate precipitation, salting out, and affinity chromatography. In some embodiments, when the BsAb provided herein comprises an immunoglobulin Fc region (e.g., QLB-1005-02000), then protein A can be used as an affinity ligand, depending on the species and isotype of the Fc region that is present in the BsAb.

Anti-CD3×CD20 BsAb Compositions

The present disclosure also provides compositions comprising the anti-CD3×CD20 BsAb provided herein. In some embodiments, the composition is a pharmaceutical composition comprising the anti-CD3×CD20 BsAb disclosed herein and a pharmaceutically acceptable carrier.

Pharmaceutically acceptable carriers for use in the pharmaceutical compositions disclosed herein are known in the art and may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, and/or other non-toxic auxiliary substances.

To further illustrate, pharmaceutically acceptable carriers may include, for example, buffers such as phosphate, citrate, and other organic acids, antioxidants including ascorbic acid and methionine, preservatives (such as hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), proteins such as serum albumin, gelatin, or immunoglobulins, hydrophilic polymers such as polyvinylpyrrolidone, amino acids such as glycine, glutamine, asparagine, histidine, arginine, or lysine, monosaccharides, disaccharides, and other carbohydrates including glucose, mannose, or dextrins, chelating agents such as EDTA, sugars such as sucrose, mannitol, trehalose or sorbitol, and/or non-ionic surfactants such as TWEEN™ or polyethylene glycol (PEG).

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, sodium saccharine, cellulose, and/or magnesium carbonate.

In some embodiments, the pharmaceutical compositions are formulated into an injectable composition. The injectable pharmaceutical compositions may be prepared in any conventional form, such as liquid solution, suspension, emulsion, or solid forms suitable for generating a liquid solution, suspension, or emulsion.

In some embodiments, unit-dose parenteral preparations of the pharmaceutical compositions are packaged in an ampoule, a vial, or a syringe with a needle. All preparations for parenteral administration should be sterile, as is known and practiced in the art. Sterilization can be accomplished by filtration through suitable filtration membranes.

In some embodiments, a sterile, lyophilized powder is prepared by dissolving the CD3×CD20 BsAb disclosed herein in a suitable solvent. The solvent may contain an excipient such as water, dextrose, sorbital, fructose, corn syrup, xylitol, glycerin, glucose, sucrose, or other suitable agent. The solvent may contain a buffer, such as citrate, sodium, potassium phosphate, or other such buffer known to those of skill in the art. The lyophilized powder can be stored under appropriate conditions, such as at about 4° C. to room temperature. Reconstitution of a lyophilized powder with water for injection provides a pharmaceutical composition for use in parenteral administration.

Methods of Treatment

The present disclosure provides for methods for treating a subject suffering from a cancer or a tumor, comprising administering to the subject a therapeutically effective amount of the CD3×CD20 BsAb disclosed herein. In some embodiments, the cancer is a B cell-related cancer.

In some embodiments, efficacy of the methods for treatment provided herein are assessed by examining cancer response. Cancer response can be assessed by examining changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In some embodiments, the B cell-related cancer includes Hodgkin's lymphoma, non-Hodgkin's lymphoma, precursor B cell lymphoblastic leukemia/lymphoma, mature B cell neoplasms, B cell chronic lymphocytic leukemia, small lymphocytic lymphoma, B cell prolymphocytic leukemia, lymphoplasmacytic lymphoma, mantle cell lymphoma, follicular lymphoma, cutaneous follicle center lymphoma, marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma.

A therapeutically effective amount of the CD3×CD20 BsAb disclosed herein may vary according to factors such as the disease state, age, sex, and weight of the subject and the ability of the medicaments to elicit a desired response in the subject. A therapeutically effective amount is also one in which any toxic or detrimental effects of the antibody are outweighed by the therapeutically beneficial effects. Dosages may be proportionally reduced or increased by one of ordinary skill in the art (e.g., physician).

An exemplary, non-limiting range for a therapeutically effective amount of the CD3×CD20 BsAb disclosed herein is about 0.01 mg/kg to about 100 mg/kg, for example, about 0.01 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, the CD3×CD20 BsAb disclosed herein is administered at a dosage of about 50 mg/kg or less, about 10 mg/kg or less, about 5 mg/kg or less, about 3 mg/kg or less, about 1 mg/kg or less, about 0.5 mg/kg or less, or about 0.1 mg/kg or less. In certain embodiments, the administration dosage may change over the course of treatment. A medical professional having ordinary skill in the art may readily determine and prescribe the effective amount of the pharmaceutical composition required. For example, a physician could start doses at levels lower than that required in order to achieve the desired therapeutic effect and gradually increase the dosage until the desired effect is achieved.

The CD3×CD20 BsAb disclosed herein may be administered by any route known in the art, such as parenteral (e.g., subcutaneous, intraperitoneal, intravenous, intramuscular, or intradermal injection) or non-parenteral (e.g., oral, intranasal, intraocular, sublingual, rectal, or topical) routes. A medical professional having ordinary skill in the art may readily determine the appropriate dosing route.

In some embodiments, the CD3×CD20 BsAb disclosed herein (for example, QLB1005-0002) is useful for treating a subject who has developed resistance to anti-CD20 therapy, for example, a subject who has developed resistance to rituximab therapy. Analytic and/or diagnostic methods known in the art, such as tumor scanning, may be used to ascertain whether a patient has developed resistance to anti-CD20, e.g., rituximab, therapy. Thus, in some embodiments, the present disclosure provides for methods for treating a subject suffering from a B cell-related cancer, comprising administering the CD3×CD20 BsAb disclosed herein 1 week, 2 weeks, 3 weeks, 4 weeks, 2 months, 4 months, 6 months, 8 months, 1 year, 2 years, or more after the subject has received anti-CD20 therapy (e.g., rituximab treatment). At the start of the administration of the CD3×CD20 BsAb disclosed herien, the subject may have developed resistance to prior anti-CD20 therapy.

In some embodiments, the treatment disclosed herein results in significant or complete cancer remission, even in a subject who has developed resistance to anti-CD20, e.g., rituximab, therapy. For example, with respect to significant cancer remission, the subject may show significantly reduced tumors with normalization of any previously abnormal radiographic studies. With respect to complete cancer remission, the subject may show an absence of clinically detectable tumors with normalization of any previously abnormal radiographic studies. A medical professional having ordinary skill in the art may readily determine significant or complete cancer remission.

### EXAMPLES

### Example 1: Bispecific Anti-CD3×CD20 Antibodies

1.1 Antibody Design and Engineering

Three anti-CD3×CD20 BsAbs were developed (FIG. 1). Among them, two antibodies comprise the NCl/NIH CH1-CK stabilized Fab format (see, e.g., US 2018/0118815 A1; Chen et al., mAbs, 2016, 8(4):761-774), and the third antibody comprises an Fc format containing Knob-in-Hole (KIH) technology. In addition, to reduce the potential effector functions of the Fc region, L234A, L235A (LALA) mutations were introduced to the CH2 domains of human IgG1 Fc region.

The scFv for anti-CD3 was derived from the humanized SP34 (ATCC) clone developed by Silvana Passano et al. in 1985 (EMBO J., 1985, 4(2):337-344). The scFv for anti-CD20 was derived from humanized rituximab (Roche). The antibody humanization protocol used in this example utilized CDR grafting in combination with protein resurfacing technology, all of which are known in the art and routinely practiced.

During the initial antibody design and gene synthesis process, three versions of the Fab format and four versions of the Fc format were obtained. Preliminary results showed that two scFv versions and one scFv-Fc version (FIG. 1) have good developmental potential. The major difference between the two Fab formats (QLB1005-0001 and QLB1005-0003) is the order of the variable domains: VH-VL-CK/VH-VL-CH1 for QLB1005-0003 and VL-VH-CH1NL-VH-CK for QLB1005-0001. The Fc format QLB1005-0002 maintains the same variable domain order as QLB1005-0003 but does not contain the stabilized CK/CH1 region. Instead, QLB1005-0002 contains a human IgG1 Fc region having LALA mutations known in the art.

Table 3 lists the linker sequences for these three antibodies. The linkers between VH and VL are the same for both Fab and Fc formats and have the sequence of SEQ ID NO: 13. The linkers between Fvs and CH1/CK in the Fab format have the sequence of SEQ ID NO: 14. The linkers between Fv and CH2 domains in the Fc format have the sequence of SEQ ID NO: 19, which contains the sequence of SEQ ID NO: 13 plus the wild type IgG1 hinge region (PKSCDKTHTCPPC; SEQ ID NO: 26).

Table 3. Linker Sequences.

TABLE 3
Linker sequences.

| Antibody | Linker (VH-VL) | Linker (scFV-CH/Fc) |
|---|---|---|
| QLB1005-0001 | SEQ ID NO: 13 | SEQ ID NO: 14 |
| QLB1005-0002 | SEQ ID NO: 13 | SEQ ID NO: 19 |
| QLB1005-0003 | SEQ ID NO: 13 | SEQ ID NO: 14 |

1.2 Sequences of Anti-CD3×CD20 BsAbs

The QLB1005-0001 antibody sequence is provided below with the CDR sequences annotated in bold and underline.

Anti-CD3 Chain of QLB1005-0001 Antibody

(SEQ ID NO: 15)
QAVVTQEPSLTVSPGGIVTLTCRSSTGAVTTSNYANWVQQKPGQAPRGLI
GGTNKRAPGVPARFSGSLLGDKAALTLSGAQPEDEAEYYCALWYSNLWVF
GTGTKVTVLGGGGSGGGGSGGGGSEVQLVESGGGLVQPGGSLKLSCAASG
FTFNTYAMNWVRQASGKGLEWVARIRSKYNNYATYYADSVKDRFTISRDD
SKNTLYLQMNSLKTEDTAVYYCVRHGNFGNSYVSWFAYWGQGTLVTVSSG
GGGSASTKGPSVFPLAPSSKSTSGGTAALGCLVKDYFPEPVTVSWNSGAL
TSGVHTFPAVLQSSGLYELVSVVTVPSSSLGTQTYICNVNHKPSNTKVDK
KVEPKSC

Anti-CD20 Chain of QLB1005-0001 Antibody

(SEQ ID NO: 16)
QIVLTQSPATLSLSPGERATLSCRASSSVSYIHWFQQKPGQAPRPWIYAT
SNLASGVPARFSGSGSGTDYTLTISSLEPEDFAVYYCQQWTSNPPTFGQG
TKVEIKGGGGSGGGGSGGGGSQVQLVQSGAEVKKPGASVKVSCKASGYTF
TSYNMHWVRQAPGQGLEWIGAIYPGNGDTSYNQKFKGKATLTADKSTSTA
YMELSSLRSEDTAVYYCARSTYYGGDWYFNVWGQGTLVTVSSGGGGSRTV
AAPSVFIFPPSDEQLKSGTASVVCLLNNFYPREAKVQWKVDNALQSGNSQ
ESVTEQDSKDSTYSLLSSLTLSKADYEKHKVYACEVTHQGLSSPVTKSFN
RGEC

The QLB1005-0002 antibody sequence is provided below with the CDR sequences annotated in bold and underline.

Anti-CD3 Chain of QLB1005-0002 Antibody

(SEQ ID NO: 20)
EVQLVESGGGLVQPGGSLKLSCAASGFTFNTYAMNWVRQASGKGLEWVAR
IRSKYNNYATYYADSVKDRFTISRDDSKNTLYLQMNSLKTEDTAVYYCVR
HGNFGNSYVSWFAYWGQGTLVTVSSGGGGSGGGGSGGGGSQAVVTQEPSL
TVSPGGTVTLTCRSSTGAVTTSNYANWVQQKPGQAPRGLIGGTNKRAPGV
PARFSGSLLGDKAALTLSGAQPEDEAEYYCALWYSNLWVFGTGTKVTVLG
GGGSGGGGSGGGGSPKSCDKTHTCPPCPAPEAAGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALGAPIEKTISKAKGQPREPQVCTLP
PSRDELTKNQVSLSCAVKGFYPSDIAVEWESNGQPENNYKTTPPVLDSDG
SFFLVSKLTVDKSRWQQGNVFSCSVMHEALHNHYTQKSLSLSPGK

Anti-CD20 Chain of QLB1005-0002 Antibody

(SEQ ID NO: 21)
QVQLVQSGAEVKKPGASVKVSCKASGYTFTSYNMHWVRQAPGQGLEWIGA
IYPGNGDTSYNQKFKGKATLTADKSTSTAYMELSSLRSEDTAVYYCARST
YYGGDWYFNVWGQGTLVTVSSGGGGSGGGGSGGGGSQIVLTQSPATLSLS
PGERATLSCRASSSVSYIHWFQQKPGQAPRPWIYATSNLASGVPARFSGS
GSGTDYTLTISSLEPEDFAVYYCQQWTSNPPTFGQGTKVEIKGGGGSGGG
GSGGGGSPKSCDKTHTCPPCPAPEAAGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALGAPIEKTISKAKGQPREPQVYTLPPCRDELT
KNQVSLWCLVKGFYPSDIAVEWESNGQPENNYKTTPPVLDSDGSFFLYSK
LTVDKSRWQQGNVFSCSVMHEALHNHYTQKSLSLSPGK

The QLB1005-0003 antibody sequence is provided below with the CDR sequences annotated in bold and underline.

Anti-CD3 Chain of QLB1005-0003 Antibody

(SEQ ID NO: 17)
EVQLVESGGGLVQPGGSLKLSCAASGFTFNTYAMNWVRQASGKGLEWVAR
IRSKYNNYATYYADSVKDRFTISRDDSKNTLYLQMNSLKTEDTAVYYCVR
HGNFGNSYVSWFAYWGQGTLVTVSSGGGGSGGGGSGGGGSQAVVTQEPSL
TVSPGGTVTLTCRSSTGAVTTSNYANWVQQKPGQAPRGLIGGTNKRAPGV
PARFSGSLLGDKAALTLSGAQPEDEAEYYCALWYSNLWVFGTGTKVTVLG
GGGSASTKGPSVFPLAPSSKSTSGGTAALGCLVKDYFPEPVTVSWNSGAL
TSGVHTFPAVLQSSGLYELVSVVTVPSSSLGTQTYICNVNHKPSNTKVDK
KVEPKSC

Anti-CD20 Chain of QLB1005-0003 Antibody

(SEQ ID NO: 18)
QVQLVQSGAEVKKPGASVKVSCKASGYTFTSYNMHWVRQAPGQGLEWIGA
IYPGNGDTSYNQKFKGKATLTADKSTSTAYMELSSLRSEDTAVYYCARST
YYGGDWYFNVWGQGTLVTVSSGGGGSGGGGSGGGGSQIVLTQSPATLSLS
PGERATLSCRASSSVSYIHWFQQKPGQAPRPWIYATSNLASGVPARFSGS
GSGTDYTLTISSLEPEDFAVYYCQQWTSNPPTFGQGTKVEIKGGGGSRTV
AAPSVFIFPPSDEQLKSGTASVVCLLNNFYPREAKVQWKVDNALQSGNSQ
ESVTEQDSKDSTYSLLSSLTLSKADYEKHKVYACEVTHQGLSSPVTKSFN
RGEC

### Example 2: In Vitro Functional Assessment of Anti-CD3×CD20 BsAbs

Lymphocytes, including CD4⁺ and CD8⁺ T cells, play a major role in cancer immune surveillance and tumor growth control. CD3, part of the T cell receptor (TCR) complex, is a pan T cell surface marker and acts as the signal transduction molecule in TCR mediated T cell activation and apoptosis. Cross-linking by mAbs specific for CD3 delivers signals to T cells, which will either activate or deplete the T cells depending on the epitopes and the affinity of the mAbs. The anti-CD3×CD20 BsAbs according to this disclosure were designed to moderately activate T cells without any significant depletion effects. At the same time, these BsAbs will bring T cells closer to tumor cells that express CD20. CD20 is a multi-span transmembrane molecule expressed on the B cell lineage. Many hematologic cancers, including lymphomas, myelomas, and B cell leukaemias, have elevated CD20 expression. Thus, the CD3×CD20 BsAbs disclosed herein are useful for treating patient populations having these and other CD20+ hematologic malignancies.

To test the specificity of the CD3×CD20 BsAbs described herein, several immunological methods were performed, as described below, using humanized SP34 mAb and rituximab as control antibodies.

2.1 Cell Surface Binding of Anti-CD3×CD20 BsAbs by FACS Analysis

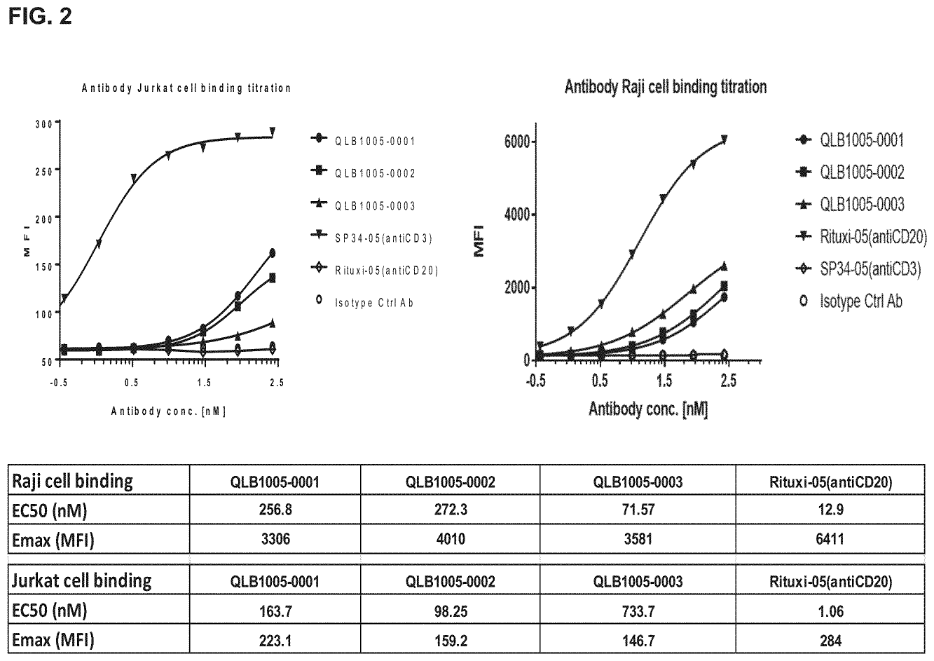

To test the cell surface binding of the anti-CD3×CD20 BsAbs disclosed herein, Jurkat cells (a CD3⁺ T cell leukemia cell line) and Raji cells (a CD20⁺ B cell lymphoma cell line) were chosen as the target cells. For fluorescence staining, the target cells were incubated with different concentrations of anti-CD3×CD20 BsAbs or control antibodies. After washing in phosphate buffered saline (PBS), fluorescence conjugated secondary antibodies were added to the cell mixture. The stained cells were analyzed in an LSR II FACS machine (BD Biosciences). Mean fluorescence intensity (MFI) was used to indicate the binding strength of the tested antibodies. FIG. 2 shows the cell surface binding patterns for the anti-CD3×CD20 BsAbs and control antibodies. The data demonstrated that both arms of the anti-CD3×CD20 BsAbs bind to their corresponding antigens on the target cells in a dose-dependent manner. The anti-CD3×CD20 BsAbs according this disclosure showed weaker CD3 arm binding (see, e.g., QLB1005-02000: 98.25 nM) in comparison to the control monoclonal anti-CD3 Ab (humanized SP34: 1.06 nM). Similar patterns were seen in the anti-CD20 arm (CD20 binding to Raji cells). A weaker CD3 binding is expected to have fewer side effects due to high affinity binding and depletion of T cells. Isotype control and cross binding control (CD3 arm to Raji cells and CD20 arm to Jurkat cells) showed no binding, demonstrating the high specificity of all three anti-CD3×CD20 BsAbs.

2.2 Functional Activity of Anti-CD3×CD20 BsAbs

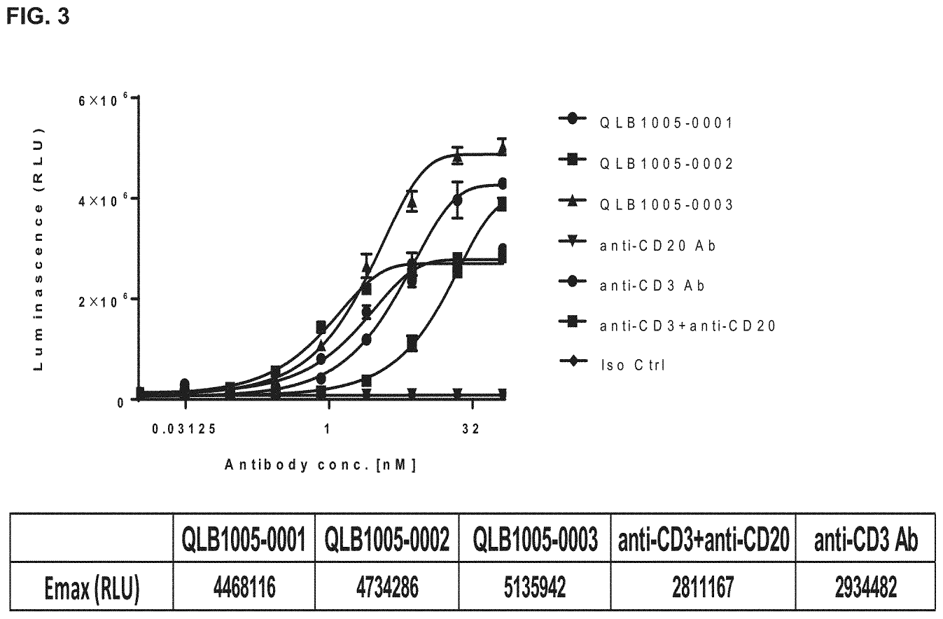

A luciferase reporter assay was used to assess the functional activity of the anti-CD3×CD20 BsAbs disclosed herein. The CD3 effector cells were engineered to express luciferase that was under the control of the TCR/CD3 downstream signal pathway. In the presence of CD20⁺ tumor target cells and the anti-CD3×CD20 BsAbs, the TCR/CD3 complex will deliver a signal to activate luciferase expression, which is then detected by chemiluminescence instruments (light emitted by luciferase substrate). FIG. 3 shows the functional activities of the anti-CD3×CD20 BsAbs as assessed by this luciferase reporter assay. The results showed that the anti-CD3×CD20 BsAbs delivered a much stronger T cell activation signal (max effective: 4.5-5.1 M RLU) compared to anti-CD3 antibody alone (2.9 M RLU) or a direct mixture of anti-CD3 and CD20 antibodies (2.8 M RLU).

2.3 T Cell Proliferation Induced by Anti-CD3×CD20 BsAbs in Human Peripheral Blood Mononuclear Cells

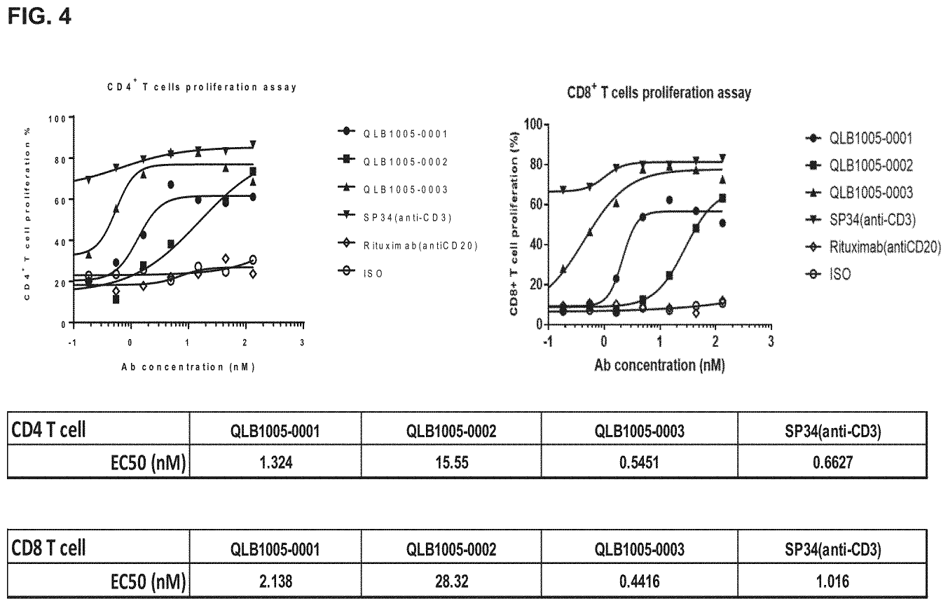

Cross-linking CD3 on the T cell surface will deliver signals that either activate or induce apoptosis of the T cells. One indicator for T cell activation is cell proliferation. To test the effects of the anti-CD3×CD20 BsAbs disclosed herein on T cell proliferation, human peripheral blood mononuclear cells (hPBMCs) were labeled with carboxyfluorescein succinimidyl ester (CFSE) for 20 minutes. After washing twice, the cells were seeded at 5×10⁵ cell/well in a 96-well plate. The anti-CD3×CD20 BsAbs disclosed herein and anti-CD3 or anti-CD20 control antibodies were added at various concentrations to the cells and incubated for 6 days. The cell culture media was supplemented with 10 ng/ml of human IL-2. After the incubation, the cells were harvested and further stained with PE labeled anti-CD8 or APC labeled anti-CD4 antibodies, and T cell proliferation was evaluated by flow cytometry monitoring of CFSE-positive cells. The percentage of proliferation was calculated based on CFSE-positive cells without antibody stimulation compared to the controls. FIG. 4 shows the stimulatory effects of the anti-CD3×CD20 BsAbs disclosed herein on human CD4⁺ and CD8⁺ T cells. In the presence of humanized SP34 mAb, both CD4⁺ and CD8⁺ T cells showed strong proliferation. All three anti-CD3×CD20 BsAbs induced dose-dependent proliferation, with EC50 ranging from 0.55 nM to 28.3 nM. Meanwhile, as expected, isotype control and rituximab did not show any proliferation induction activities.

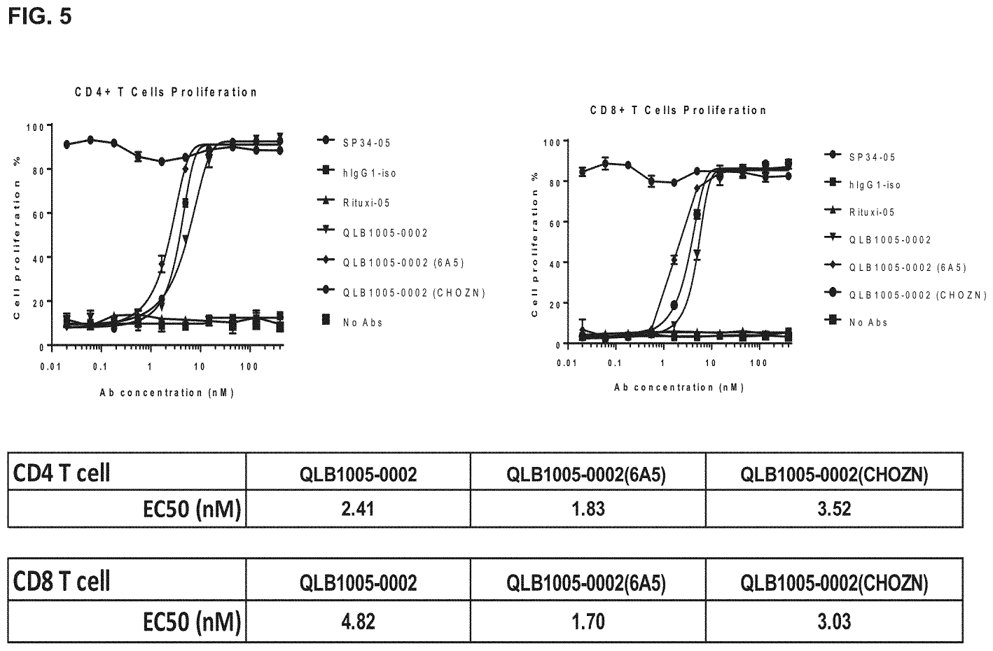

FIG. 5 further confirmed the stimulatory effects for human CD4+ and CD8⁺ T cells by QLB1005-0002, using QLB1005-0002 compositions produced from different expression systems, namely transient expression, CHOZN stable pool, and stable single clone 6A5.

2.4 Tumor Cell Killing Activities by Anti-CD3×CD20 BsAbs

T lymphocytes, particularly CD8⁺ T cells, have the capability to kill target tumor cells. When drawn closer to target tumor cells, CD8⁺ T cells show more intensive killing activities on those target cells. Two CD20⁺ tumor cell lines were used to evaluate the killing activities of the anti-CD3×CD20 BsAbs disclosed herin. Briefly, Daudi and Raji tumor cells (target cells) were labeled with CellTrace® CFSE (Thermo Fisher). The antibody concentration was prepared at 133 nM with 3× titration, and the tumor cells were seeded at 40,000 per well and co-cultured for 24 hours with hPBMCs (effector cells) in the presence of the anti-CD3×CD20 BsAbs disclosed herein and control antibodies. The ratio of the effector and target cells was at 10:1 in this assay. After 24 h incubation, the cells were centrifuged, washed twice with PBS and then stained with cell live/dead marker 7-AAD (Invitrogen™ eBioscience™ 7-AAD, cat #50-112-8859). The dead tumor cells were measured by flow cytometer. The killing activity was calculated based on the ratio of killing with/without the anti-CD3×CD20 BsAbs or control antibodies.

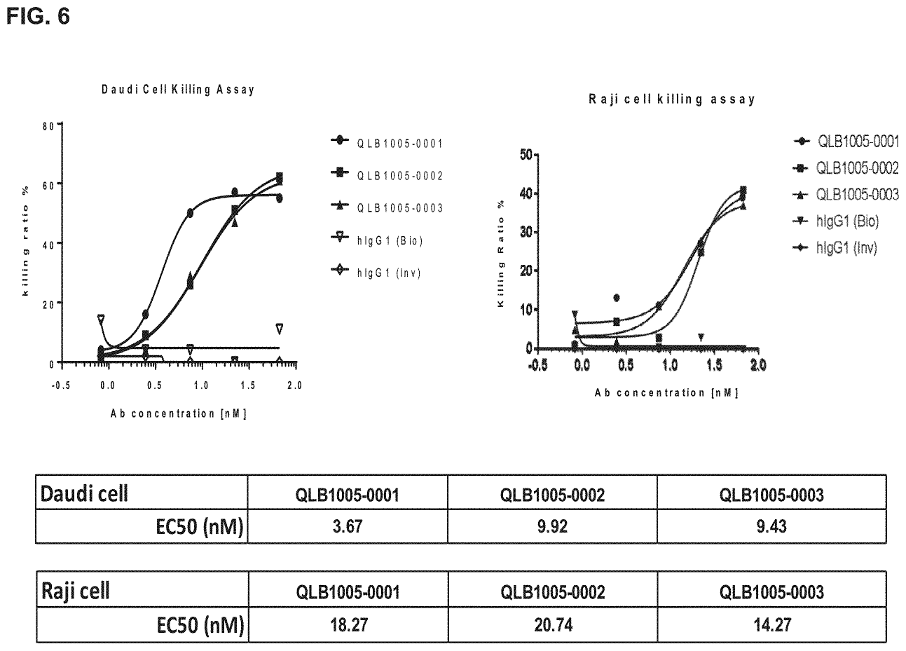

As shown in FIG. 6, the anti-CD3×CD20 BsAbs, but not control antibodies, demonstrated dose-dependent T cell killing activities in both Raji and Daudi cells.

2.5 T Cell Killing Activity on Rituximab Resistant Tumor Cells by Anti-CD3×CD20 BsAbs

It was reported that some B lymphoma/leukemia patients develop resistance to rituximab treatments. The down-regulation of CD20 expression on those tumor cells has been considered as a major reason for the resistance to rituximab treatment. The following examples demonstrate that the anti-CD3×CD20 BsAbs according to this disclosure can be used to treat CD20 low expression tumor cells by bringing active-killing T cells closer to target cancer cells, thereby achieving better tumor-killing efficacy on rituximab resistant tumors.

2.5.1 Generation of Rituximab Resistant Raji and Daudi Cell Lines

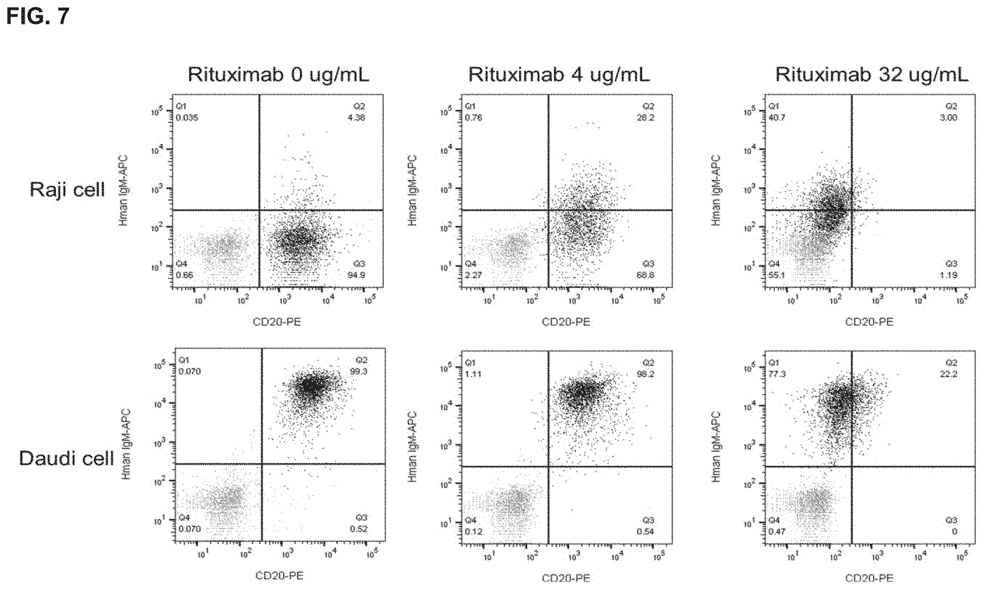

Raji and Daudi cells were cultured in rituximab-containing media to induce resistance. Specifically, Raji or Daudi cells (2 flask for each cell line, 4107/flask) were incubated at 37° C., 5% CO₂ and serially exposed for 24 h to an escalating dose of rituximab (0.125-128 μg/mL) in the presence of 5% human serum. Following the 24 h incubation, cells were centrifuged, and the medium was replaced with fresh RPMI 1640. Cells were then incubated for an additional 1-3 days until their growth reached log phase. The procedure was repeated for a total of 11 times at which time functional assays showed maximal inhibition of rituximab-associated biological activities. Functional cell killing assays with rituximab or the anti-CD3×CD20 BsAbs disclosed herein were performed every 2-3 passages. As shown in FIG. 7, the resulting cell lines showed decreased expression of CD20 and increased expression of IgM, which matched the criteria of rituximab resistant cells.

2.5.2 T Cell Killing of Rituximab Resistant Cells by Anti-CD3×CD20 BsAbs

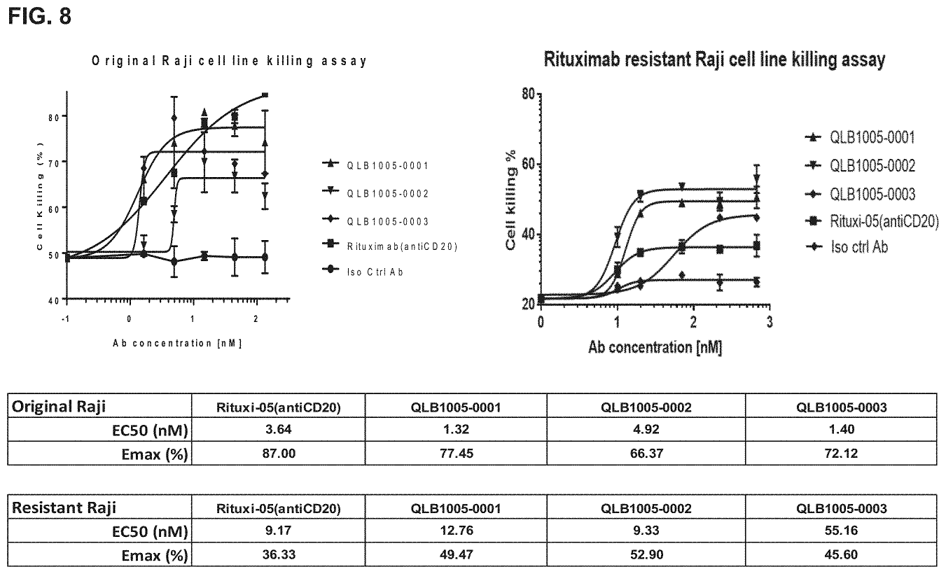

The T cell killing assay described in section 2.4 was used to assess the killing of rituximab resistant Raji and Daudi cells by the anti-CD3×CD20 BsAbs disclosed herein. The antibody concentration was prepared at 133 nM with 3× titration, and the rituximab resistant tumor cells were seeded at 40,000 per well. The treated target cells were co-cultured with hPBMCs for 24 hours, and the tumor cell killing activity was measured by flow cytometer. Compared to the original Raji and Daudi cells, the anti-CD3×CD20 BsAbs disclosed herein, but not rituximab, retained the killing activity on the rituximab resistant Raji cells (FIG. 8) and Daudi cells. In particular, the data showed a stronger killing activity for QLB1005-02000 (about 52.9%) than rituximab (about 36.3%) in killing the rituximab resistant Raji cells. In comparison, rituximab showed about 87% activity in killing the original Raji cells.

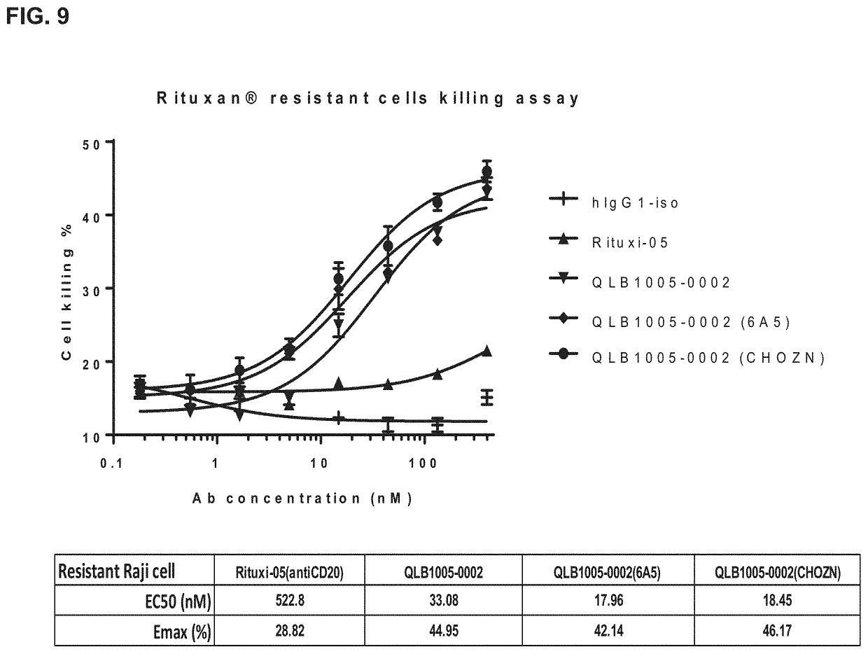

The T cell killing assay was also performed to test whether the QLB1005-02000 stable pool (CHOZN) and stable single clone 6A5 could mediate the killing of the rituximab resistant tumor cell lines. As shown in FIG. 9, both the QLB1005-0002 stable pool (QLB1005-0002 CHOZN) and the stable clone (QLB1005-0002 6A5) demonstrated similar dose-dependent activity in killing the rituximab resistant cells (about 46.2% and 42.1%, respectively), when compared to the QLB1005-0002 from transient expression (about 45.0%). In contrast, rituximab showed only about 28.8% activity in killing the rituximab resistant Raji cells.

### Example 3: In Vivo Efficacy of Anti-CD3×CD20 BsAbs in hPBMC Humanized Mouse Models

The in vivo anti-tumor effects of the anti-CD3×CD20 BsAbs disclosed herein were evaluated in Raji, rituximab resistant Raji and Daudi Cell derived xenograft (CDX) in hPBMC humanized NOD-scid IL2Rgamma^{null} (NSG) mice. In addition, a dose-response study was performed in the Raji CDX in hPBMC humanized NSG mice to determine the effective dose of QLB1005-0002.

3.1 Effects of Anti-CD3×CD20 BsAbs on Raji CDX Growth in hPBMC Humanized NSG Mice

3.1.1 Methods

Xenograft. On day 0, 3.5×10⁶ Raji cells in 0.1 mL sterile PBS were inoculated subcutaneously (s.c.) into the right flank of the NSG mice (6-8 weeks, Jackson Laboratory, Bar Harbor, Me.). On day 3, 1×10⁷ hPBMCs were injected intraperitoneally (i.p.) into the NSG mice. The hPBMCs were from 2 different healthy donors labeled as donor 1 and donor 2. When the average tumor volume reached 30-50 mm³, the mice were randomized into 6 groups with matching tumor sizes (5 mice per group per donor). The anti-CD3×CD20 BsAbs disclosed herein were dosed as described in Table 4. A total of five doses were administered.

TABLE 4
Treatment groups for in vivo efficacy
studies of anti-CD3 × CD20 BsAbs

| | | | | | No. of | No. of |
|---|---|---|---|---|---|---|
| | | Dose | Sched- | Dosing | mice | mice |
| Group | Antibody | (mg/kg) | ule | route | Donor 1 | Donor 2 |
| 1 | Vehicle control | — | BIW | i.p. | 5 | 5 |
| 2 | QLB1005-0001 | 3 | BIW | i.p. | 5 | 5 |
| 3 | QLB1005-0001 | 0.3 | BIW | i.p. | 5 | 5 |
| 4 | QLB1005-0002 | 3 | BIW | i.p. | 5 | 5 |
| 5 | QLB1005-0003 | 3 | BIW | i.p. | 5 | 5 |

Body weight and tumor volume (TV) were measured 3 times a week using an electronic caliper. TV was calculated using the formula: TV (mm³)=0.5×Length×Width². The anti-tumor efficacy was expressed as Tumor Growth Inhibition (TGI), which was calculated for each group using the formula: TGI (%)=[1-(Tᵢ−T₀)/(Vᵢ−V₀)]×100, where Tᵢ is the average tumor volume of a treatment group on a given day, To is the average tumor volume of the treatment group on the first day of treatment, Vᵢ is the average tumor volume of the vehicle control group on the same day with Tᵢ, and V₀ is the average tumor volume of the vehicle group on the first day of treatment. Animals with no palpable tumors were considered as Complete Remission (CR).

Raji CDX Rechallenge. Eight days post the last dose, the original Raji CDXs were removed by surgery and the mice were rechallenged. Specifically, 4×10⁶ Raji cells were inoculated s.c. into the left flanks of all 10 QLB1005-0002-treated mice and 5 vehicle control-treated mice. Tumors on the left flanks were measured on day 6 and 8 post rechallenge.

Flow Cytometry. Human leucocyte reconstitution level was evaluated by flow cytometry. Mouse blood and spleen were processed into single cell suspensions and stained with anti-human CD45 (PE/Cy5, BioLegend), anti-human CD3 (FITC, BioLegend), and anti-human CD8 (PE, BioLegend). Samples were then processed by BD™ LSR II Flow Cytometer and analyzed by FlowJo.

Statistics. For comparison among three or more groups, one-way ANOVA was performed. If a significant F (ratio of treatment variance to the error variance) was obtained, multiple comparison procedures were applied after ANOVA. For comparison between 2 groups, Mann-Whitney Test was performed. All data were analyzed using GraphPad Prism 8.0.1. P<0.05 is statistically significant. 3.1.2 Results

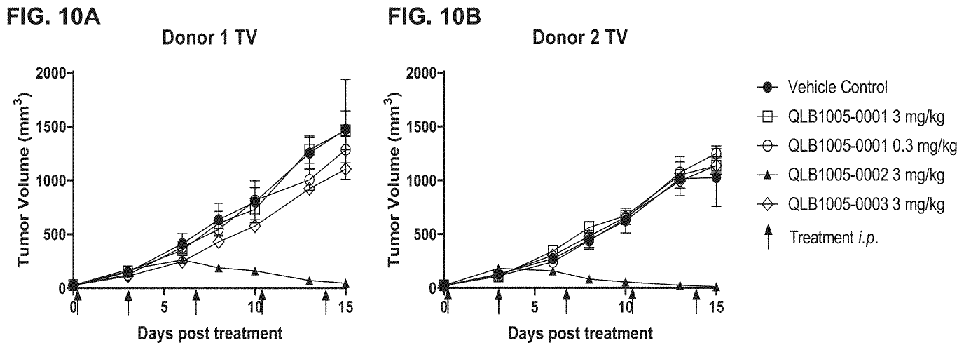

QLB1005-0002 significantly induced Raji CDX remission in hPBMC humanized NSG mice. To identify if any of the three anti-CD3×CD20 BsAbs disclosed herein has in vivo efficacy, a total of 5 doses of QLB1005-0001, QLB1005-0002, QLB1005-0003 or PBS (vehicle control) were administered into hPBMC reconstituted NSG mice with Raji CDX at a dosage of 3 mg/kg (Table 4). QLB1005-0001 was also dosed at 0.3 mg/kg (Table 4). As shown in FIGS. 10A-10B and Table 5, on day 13 post treatment, QLB1005-0002 showed significant tumor remission with TGI of 96% and 100% for hPBMC donor 1 and donor 2, respectively, whereas QLB1005-0001 and QLB1005-0003 did not show significant efficacy against Raji CDX. This result indicates that QLB1005-0002 is effective for inhibiting Raji CDX growth in vivo.

TABLE 5
Tumor Growth Inhibition (TGI) and
tumor size on day 13 post treatment
Donor 1

| | Tumor volume | | |
|---|---|---|---|
| Treatment (mg/kg) | (mm³) ^{a} | TGI | p value ^{b} |
| Vehicle Control | 1255 | — | — |
| QLB1005-0001, 3 | 1285 | −2% | 0.9997 |
| QLB1005-0001, 0.3 | 1005 | 20% | 0.3077 |
| QLB1005-0002, 3 | 69 | 96% | <0.0001 |
| QLB1005-0003, 3 | 923 | 27% | 0.0852 |

Donor 2

| | Tumor volume | | |
|---|---|---|---|
| Treatment | (mm³) ^{a} | TGI | p value ^{b} |
| Vehicle Control | 1014 | — | — |
| QLB1005-0001, 3 | 1051 | −3.7% | 0.9985 |
| QLB1005-0001, 0.3 | 1074 | −6.0% | 0.9895 |
| QLB1005-0002, 3 | 26 | 99.8% | <0.0001 |
| QLB1005-0003, 3 | 994 | 2.1% | 0.9998 |

^{a} mean;
^{b} one-way ANOVA, compared with tumor volumes of the vehicle control.

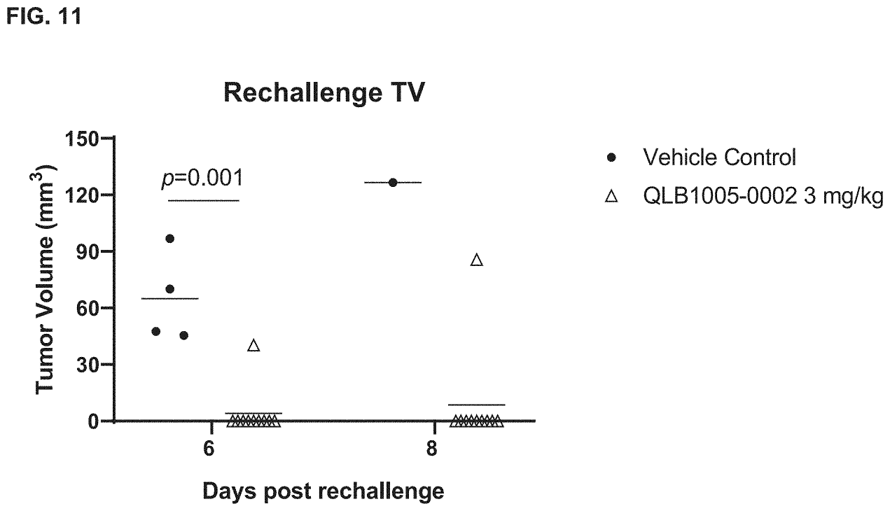

QLB1005-0002 significantly inhibited the rechallenged Raji CDX growth in hPBMC humanized NSG mice. 21 days post treatment (eight days post the last dose and one day before rechallenging), 7 out of 10 mice treated with QLB1005-0002 showed complete remission of the original tumors inoculated in the right flanks. 6 days post rechallenge of Raji cells, most mice from the QLB1005-0002 treatment group did not show any Raji CDX growth (9 out of 10 mice) while the vehicle control group showed significant tumor growth after rechallenge. On day 8 post rechallenge, only one mouse in the vehicle control group remained alive and the other mice had to be euthanized due to severe GvHD induced by the implanted hPBMC. In contrast, in the QLB1005-0002 group most CDXs remained unmeasurable (9/10) (FIG. 11). Moreover, all 10 mice treated with QLB1005-0002 showed complete remission of the original tumors at the right flanks at the end of experiment (day 30 post treatment).

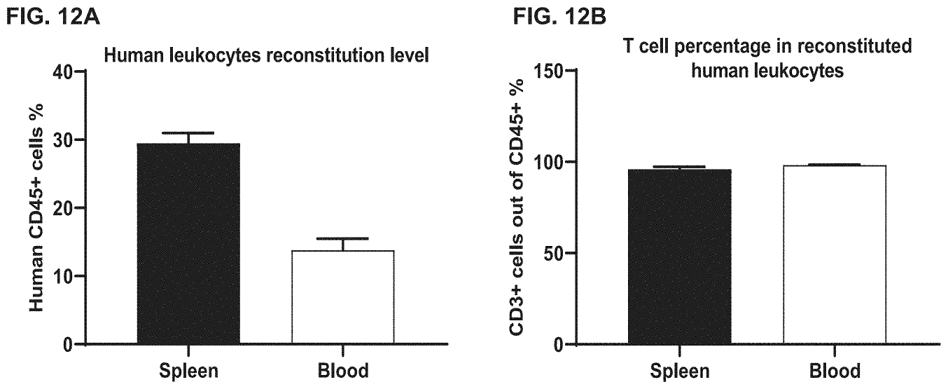

Mice injected with hPBMCs have good human leukocyte reconstitution. Flow cytometry was performed to confirm reconstitution of the human immune cells. As shown in FIG. 12A, human CD45⁺ leukocytes were well reconstituted in spleen and blood of the NSG mice. At the end point, at least 95% of CD45⁺ leukocytes were CD3⁺ T cells in blood and spleen (FIG. 12.B). 3.2 QLB1005-02000 Significantly Inhibited Rituximab Resistant Raji CDX Growth in hPBMC Humanized NSG Mice

Drug resistance is a challenge in treating B cell lymphomas and leukemias with rituximab. One of the goals for the development of the anti-CD3×CD20 BsAbs disclosed herein was to target the rituximab resistant patient population. In vitro data showed that QLB1005-0002 demonstrated enhanced T cell killing of rituximab resistant Raji cells when compared to rituximab. To test whether QLB1005-0002 is better than rituximab in inhibiting rituximab resistant Raji CDX growth in hPBMC humanized NSG mice, QLB1005-0002 and control antibodies were dosed as described in Table 6.

TABLE 6
Treatment groups for in vivo efficacy studies
of QLB1005-0002 on rituximab resistant Raji CDX

| | | Dose | | Dosing | |
|---|---|---|---|---|---|
| Group | Antibody | (mg/kg) | Schedule | route | No. of mice |
| 1 | Vehicle control | — | BIW | i.p. | 6 |
| 2 | QLB005-0002 | 3 | BIW | i.p. | 6 |
| 3 | Rituximab | 3 | BIW | i.p. | 6 |
| 4 | Anti-CD3 | 3 | BIW | i.p. | 6 |
| 5 | Anti-CD3 + | 3 | BIW | i.p. | 6 |
| | rituximab | | | | |

3.2.1 Method

Xenograft. The rituximab resistant Raji CDX in hPBMC humanized NSG mice were developed following the method described in section 3.1. This experiment used one hPBMC donor labeled as donor 3. When the average tumor volume reached 70 mm³, mice were randomized into 5 groups with matching tumor sizes (6 mice per group). A total of 6 doses were administrated i.p. twice a week. Tumor volumes were measured and calculated following the protocols described in 3.1.

3.2.2 Results

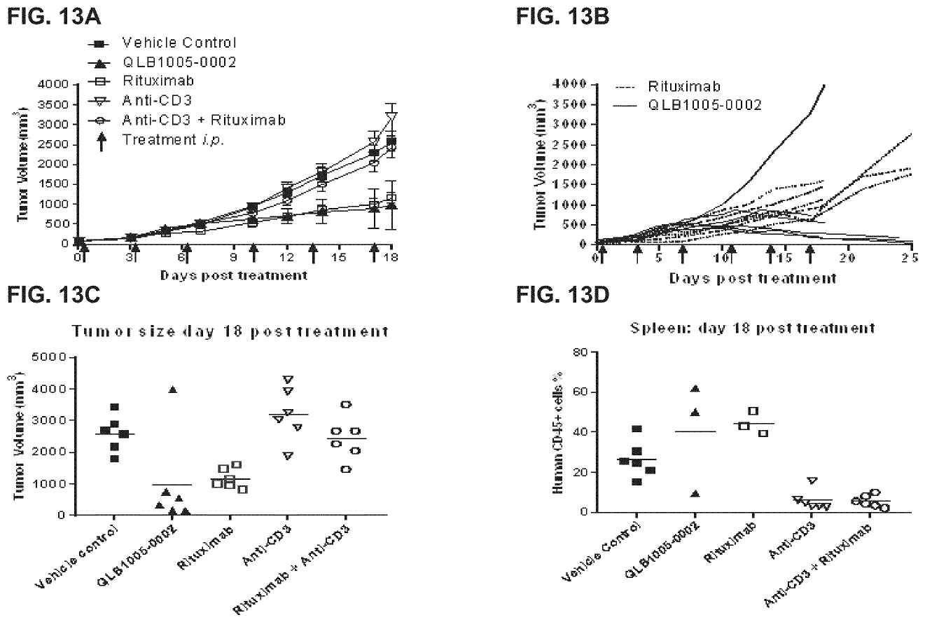

QLB1005-0002 significantly inhibited the growth of Rituximab resistant Raji CDX in hPBMC humanized NSG Mice. Both QLB1005-0002 and rituximab inhibited the growth of rituximab resistant Raji CDX in NSG mice (FIG. 13A and FIG. 13C). However, 5 out of 6 mice treated with QLB1005-0002 showed Raji CDX regression while no regression was observed in mice treated with rituximab (FIG. 13B).

On day 18 post treatment, mice were sacrificed due to large tumor volumes, except for 3 mice each with smaller tumor volumes in rituximab and QLB1005-0002 treatment groups. These 6 mice were kept for observation until day 25 post treatment. As shown in FIG. 13B, after the treatment stopped, rituximab resistant Raji CDX in QLB1005-0002-treated mice continued to shrink significantly, while the tumors in rituximab-treated mice showed significant growth.

Vehicle control, rituximab and QLB1005-0002 treated groups showed satisfactory hPBMC reconstitution levels expressed as human CD45⁺ cell percentages in spleens from the sacrificed mice (FIG. 13D). In contrast, T cell depletion was observed in groups dosed with anti-CD3 alone or anti-CD3 in combination with rituximab (FIG. 13D).

3.3 Dose Response of QLB1005-0002

To determine the minimum effective dose of QLB1005-0002, different dosages of QLB1005-0002 were tested for inhibiting Raji CDX growth in hPBMC humanized NSG mice.

3.3.1 Method

Xenograft. The xenograft was performed following the procedure described in section 3.1. This experiment used 2 hPBMC donors numbered as donor 4 and donor 5. When the average tumor volume reached 50 mm³, the mice were randomized into 6 groups with matching tumor sizes (5 mice per group per donor). The treatments were dosed as described in Table 7.

TABLE 7
Experimental design for the dose response study of QLB1005-0002

| | | | | | No. of | No. of |
|---|---|---|---|---|---|---|
| | | Dose | Sched- | Dosing | mice | mice |
| Group | Antibody | (mg/kg) | ule | route | donor 4 | donor 5 |
| 1 | Vehicle control | — | BIW | i.p. | 5 | 5 |
| 2 | QLB1005-0002 | 3 | BIW | i.p. | 5 | 5 |
| 3 | QLB1005-0002 | 1 | BIW | i.p. | 5 | 5 |
| 4 | QLB1005-0002 | 0.3 | BIW | i.p. | 5 | 5 |
| 5 | QLB1005-0002 | 0.1 | BIW | i.p. | 5 | 5 |
| 6 | QLB1005-0002 | 0.03 | BIW | i.p. | 5 | 5 |

A total of 5 doses were administered to mice receiving hPBMCs from donor 4. For mice receiving hPBMCs from donor 5, a total of 5 doses were administered to the 3 mg/kg and 1 mg/kg treatment groups. Four doses were administered to the remaining groups due to early termination of the mice after getting severe GvHD symptoms. Tumor volumes were measured and calculated following the protocols described in 3.1.

3.3.2 Results

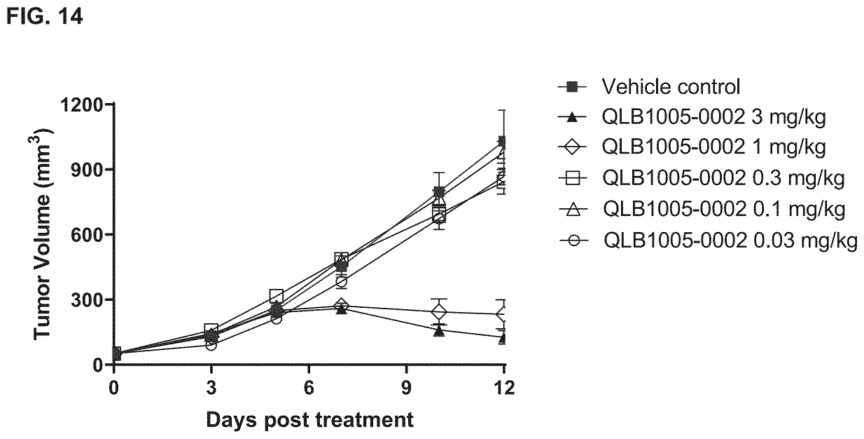

As shown in FIG. 14, mice dosed with 1 mg/kg and 3 mg/kg of QLB1005-0002 showed significant inhibition of Raji CDX growth in hPBMC reconstituted NSG mice. Furthermore, the 3 mg/kg treatment group showed better efficacy compared to the 1 mg/kg treatment group. In contrast, mice treated with 0.3 mg/kg or less of QLB1005-0002, as well as mice treated with the vehicle control, did not show significant inhibition of Raji CDX growth.

3.4 QLB1005-0002 Inhibited Daudi CDX Growth in hPBMC Humanized NSG Mice

The objective of this experiment was to test whether QLB1005-0002 could inhibit the tumor growth of another B cell lymphoma line, Daudi, CDX in hPBMC humanized NSG mice.

3.4.1 Method

Xenograft. On day 0, 7×10⁶ Daudi cells in 0.1 mL of sterile PBS were mixed with Matrigel (1:1) and were inoculated s.c. into the right flank of NSG mice. On day 3, the mice were injected i.p. with 1×10⁷ hPBMCs obtained from healthy donor labeled as donor 6. When the average tumor volume reached 140 mm³, the mice were randomized into 2 groups with matching tumor sizes (7 mice per group). 3 mg/kg of QLB1005-0002 was dosed twice a week. A total of 5 doses were administered. Tumor volumes were measured and calculated following the protocols described in 3.1.

3.4.2 Results

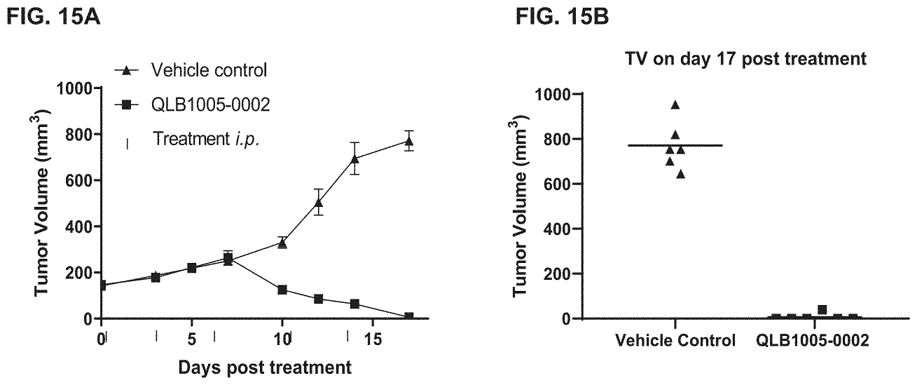

QLB1005-0002 significantly inhibited another B cell lymphoma line, Daudi, CDX growth in hPBMC humanized NSG mice. Similar to the results obtained from the Raji CDX model, treatment with 3 mg/kg of QLB1005-0002 significantly inhibited the growth of Daudi CDX in hPBMC humanized NSG mice (FIG. 15A). Furthermore, 5 out of 6 (83.3%) mice showed complete tumor remission (FIG. 15B). Thus, the data demonstrated that QLB1005-0002 significantly inhibited Daudi CDX growth in hPBMC humanized NSG mice.

3.5 QLB1005-0002 Inhibited Raji CDX Growth Even with Delayed Treatment

The objective of this experiment was to test whether QLB1005-0002 could inhibit tumor growth, when it was administered in a delayed treatment regimen. Specifically, in this experiment QLB1005-0002 was administered to the hPBMC humanized NSG mice when the average tumor size of Raji CDXs reached 300 mm³.

3.5.1 Method

Xenograft. Raji CDX in hPBMC humanized NSG mice was developed following the procedure described in section 3.1. The hPBMCs from 2 donors labeled as donor 7 and donor 8 were used. On day 14, the average tumor size reached 280-300 mm³ for both Raji CDXs. The mice were randomized into 4 groups and treatments were delivered every 3 days following the dosing as described in Table 8, with a total of 3 doses administered.

TABLE 8
Experimental design for delayed dosing of QLB1005-0002

| | | | | | No. of | No. of |
|---|---|---|---|---|---|---|
| | | Dose | Sched- | Dosing | mice | mice |
| Group | Antibody | (mg/kg) | ule | route | donor 7 | donor 8 |
| 1 | Vehicle control | — | Q3D | i.p. | 5 | 5 |
| 2 | QLB1005-0002 | 10 | Q3D | i.p. | 5 | 5 |
| 3 | QLB1005-0002 | 3 | Q3D | i.p. | 5 | 5 |
| 4 | QLB1005-0002 | 1 | Q3D | i.p. | 5 | 5 |

3.5.2 Results

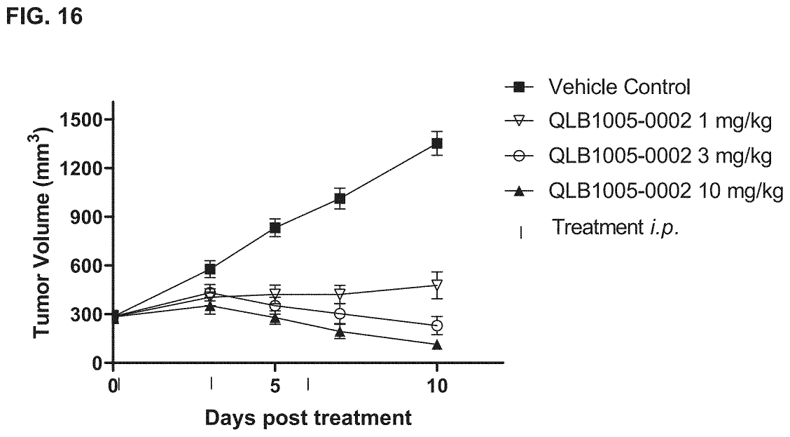

QLB1005-0002 significantly inhibited tumor growth even if the treatment started after the Raji CDXs reached the size of 300 mm³. As shown in FIG. 16, QLB1005-0002 dosed at 10 mg/kg, 3 mg/kg, and 1 mg/kg significantly inhibited Raji CDX growth. Among these treatment groups, 10 mg/kg treatment demonstrated the best efficacy. Furthermore, QLB1005-0002 showed excellent safety profile with no obvious body weight loss when administrated i.p. at the highest dose of 10 mg/kg.

### REFERENCES

All references referred to are incorporated herein by reference in their entireties.
Pallavi Bhatta and David P. Humphreys, “Relative Contribution of Framework and CDR Regions in Antibody Variable Domains to Multimerisation of Fv- and scFv-Containing Bispecific Antibodies,” Antibodies, 2018, 7(3), 35.
Eva Dahlén, Niina Veitonmäki, and Per Norlén, “Bispecific antibodies in cancer immunotherapy,” Therapeutic Advances in Vaccines and Immunotherapy, 2018, 6:3-17.
Ivana Spasevska, Minh Ngoc Duong, Christian Klein and Charles Dumontet, “Advances in Bispecific Antibodies Engineering: Novel Concepts for Immunotherapies,” J Blood Disorders Transf., 2015, 6: 243. DOI: 10.4172/2155-9864.1000243.
Weizao Chen, Ariola Bardhi, Yang Feng, Yanping Wang, Qianqian Qi, Wei Li, Zhongyu Zhu, Marzena A. Dyba, Tianlei Ying, Shibo Jiang, Harris Goldstein, and Dimiter S. Dimitrov, “Improving the CH1-CK heterodimerization and pharmacokinetics of 4Dm2m, a novel potent CD4-antibody fusion protein against HIV-1,” mAbs, 2016, 8(4):761-774.

What is claimed is:

1. A bispecific antibody comprising:
(i) a single-chain CD3 binding moiety comprising a heavy chain variable domain (VH) operably linked to a light chain variable domain (VL) via a first peptide linker, wherein
the VH comprises a heavy chain CDR1 comprising SEQ ID NO: 1, a heavy chain CDR2 comprising SEQ ID NO: 2, and a heavy chain CDR3 comprising SEQ ID NO: 3; and
the VL comprises a light chain CDR1 comprising SEQ ID NO: 4, a light chain CDR2 comprising SEQ ID NO: 5, and a light chain CDR3 comprising SEQ ID NO: 6, and
(ii) a single-chain CD20 binding moiety comprising a VH operably linked to a VL via a second peptide linker, wherein
the VH comprises a heavy chain CDR1 comprising SEQ ID NO: 7, a heavy chain CDR2 comprising SEQ ID NO: 8, and a heavy chain CDR3 comprising SEQ ID NO: 9; and
the VL comprises a light chain CDR1 comprising SEQ ID NO: 10, a light chain CDR2 comprising SEQ ID NO: 11, and a light chain CDR3 comprising SEQ ID NO: 12.

2. The bispecific antibody of claim 1, wherein
the VH in the single-chain CD3 binding moiety comprises SEQ ID NO: 22, and the VL in the single-chain CD3 binding moiety comprises SEQ ID NO: 23; and/or
the VH in the single-chain CD20 binding moiety comprises SEQ ID NO: 24, and the VL in the single-chain CD20 binding moiety comprises SEQ ID NO: 25.

3. The bispecific antibody of claim 1, wherein the first and/or second peptide linker comprises SEQ ID NO: 13.

4. The bispecific antibody of claim 1, further comprising a CH1 domain and a CK domain, wherein the CH1 domain and the CK domain forms a heterodimer.

5. The bispecific antibody of claim 4, wherein the VH in the single-chain CD3 binding moiety is operably linked to the CH1 domain via a third peptide linker, and wherein the VH in the single-chain CD20 binding moiety is operably linked to the CK domain via a fourth peptide linker.

6. The bispecific antibody of claim 5, wherein the third and/or fourth peptide linker comprises SEQ ID NO: 14.

7. The bispecific antibody of claim 6, comprising a first polypeptide sequence comprising SEQ ID NO: 15, and a second polypeptide comprising SEQ ID NO: 16.

8. The bispecific antibody of claim 4, wherein the VL in the single-chain CD3 binding moiety is operably linked to the CH1 domain via a third peptide linker, and wherein the VL in the single-chain CD20 binding moiety is operably linked to the CK domain via a fourth peptide linker.

9. The bispecific antibody of claim 8, wherein the third and/or fourth peptide linker comprises SEQ ID NO: 14.

10. The bispecific antibody of claim 9, comprising a first polypeptide sequence comprising SEQ ID NO: 17, and a second polypeptide comprising SEQ ID NO: 18.

11. The bispecific antibody of claim 1, further comprising an Fc region comprising a first and second CH2 domain and a first and second CH3 domain.

12. The bispecific antibody of claim 11, wherein the VL in the single-chain CD3 binding moiety is operably linked to the first CH2 domain via a third peptide linker, and wherein the VL in the single-chain CD20 binding moiety is operably linked to the second CH2 domain via a fourth peptide linker.

13. The bispecific antibody of claim 12, wherein the third and/or fourth peptide linker comprises SEQ ID NO: 19.

14. The bispecific antibody of claim 11, wherein the first and second CH2 domain comprises a L234A and/or L235A mutation.

15. The bispecific antibody of claim 14, comprising a first polypeptide sequence comprising SEQ ID NO: 20, and a second polypeptide comprising SEQ ID NO: 21.

16. An isolated polynucleotide encoding the single-chain CD3 binding moiety and/or the single-chain CD20 binding moiety of the bispecific antibody of claim 1.

17. A vector comprising one or more polynucleotides encoding the bispecific antibody of claim 1.

18. A composition comprising the bispecific antibody of claim 1.

19. A pharmaceutical composition comprising the bispecific antibody of claim 1 and a pharmaceutically acceptable carrier.

20. A host cell capable of expressing the bispecific antibody of claim 1.

21. A method of producing a bispecific antibody, comprising culturing the host cell of claim 20 and recovering the bispecific antibody from the host cell.

22. A method of treating a subject suffering from a B cell-related cancer, comprising administrating to the subject a therapeutically effective amount of the bispecific antibody of claim 1.

23. The method of claim 22, wherein the B cell-related cancer is selected from Hodgkin's lymphoma, non-Hodgkin's lymphoma, precursor B cell lymphoblastic leukemia/lymphoma, mature B cell neoplasms, B cell chronic lymphocytic leukemia, small lymphocytic lymphoma, B cell prolymphocytic leukemia, lymphoplasmacytic lymphoma, mantle cell lymphoma, follicular lymphoma, cutaneous follicle center lymphoma, marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma.

24. The method of claim 22, wherein the subject has developed resistance to anti-CD20 therapy.

25. The method of claim 22, wherein the subject has developed resistance to rituximab therapy.

26. The method of claim 22, wherein the treatment results in significant or complete cancer remission.
